(19) United States
(12) Patent Application Publication
(10) Pub. No.: US 2020/0397224 A1
(43) Pub. Date: Dec. 24, 2020

(54) WIRELESS VIEWING DEVICE AND METHOD OF USE THEREOF

(71) Applicant: A.M. SURGICAL, INC., Smithtown, NY (US)

(72) Inventors: Romi Mirza, Henderson, NV (US); Ather Mirza, Saint James, NY (US)

(21) Appl. No.: 17/012,572

(22) Filed: Sep. 4, 2020

Related U.S. Application Data
(63) Continuation-in-part of application No. 16/145,443, filed on Sep. 28, 2018.
Application No. 16/145,443 is a continuation-in-part of application No. 15/227,463, filed on Aug. 3, 2016.
Application No. 15/227,463 is a continuation-in-part of application No. 14/961,537, filed on Dec. 7, 2015.
(60) Provisional application No. 62/134,914, filed on Mar. 18, 2015.

(51) Int. Cl. A61B 1/00; A61B 1/05; A61B 1/06; A61B 1/005

(52) U.S. Cl. CPC A61B 1/00016; A61B 1/00022; A61B 1/00032; A61B 1/05; A61B 1/00103; A61B 1/0684; A61B 1/005; A61B 1/00009; A61B 1/00045; A61B 1/00096

(57) ABSTRACT

A wireless endoscopic viewing device comprises a housing, a shaft and a camera mounted on the tip of the shaft. The housing contains a circuit board, a transmitter, and a power source.

This application is a continuation-in-part of co-pending U.S. patent application Ser. No. 16/145,443, filed on Sep. 28, 2018 and titled “Wireless Viewing Device And Method Of Use Thereof”, which is a continuation-in-part of U.S. patent application Ser. No. 15/227,463, filed on Aug. 3, 2016 and titled “Wireless Viewing Device”, which is a continuation-in-part of U.S. patent application Ser. No. 14/961,537, filed on Dec. 7, 2015 and titled “Video Assisted Surgical Device”, which is related to U.S. Provisional Patent Application Ser. No. 62/134,914, filed on Mar. 18, 2015 and titled “Video Assisted Handheld Surgical Device”, the disclosure of each of which is incorporated herein by reference and on which priority is hereby claimed.

### FIELD

This application generally relates to medical devices. In particular, the application relates to wireless viewing devices and the use thereof.

### BACKGROUND

Conventional surgical techniques and equipment often require a fairly large incision over the surgical site and spreading of the incision to allow viewing and instrument access. These techniques can require a longer period of recovery than endoscopic methods and have greater levels of post-operative pain due to the incision size and level of manipulation during the procedure.

Endoscopic surgeries are minimally invasive surgical procedures that are performed through small incisions or natural body openings. An endoscopic procedure typically involves use of specialized devices and direct- or remote-control manipulation of instruments with indirect observation of the surgical field through an endoscope, or similar device. Compared to open surgery, endoscopic surgery is a minimally invasive surgery with less postoperative pain, early resumption of usual activities and a cosmetically appealing scar. It typically results in shorter hospital stays, or allows outpatient treatment.

In general, endoscopy is applied to introduction of a scope into a joint anywhere in the body. Endoscopic surgery refers to the process of introducing of the instrument to, and performing an operation at the joint. Endoscopy is applied to introduction of a scope into a body cavity anywhere in the body. Endoscopic surgery refers to the process of introducing the instruments and performing surgery at the operation site. Further nomenclature is designated by the anatomical structure the scope is introduced into, for example if the scope is placed in the stomach it is called Gastroscopy, in the abdomen it is Laparoscopy, etc. There are places where no actual cavity exists. Here, surgeons can create a cavity by introducing a slotted cannula to visualize the surroundings without soft tissue obstruction, as in endoscopic carpal tunnel and cubital tunnel.

As seen in recent outbreaks of infections in hospitals, the insufficient or improper sterilization of re-usable surgical implements can result in the introduction of microorganisms, including drug-resistant bacteria, into the patient, potentially resulting in severe, or even lethal, infections. This risk is magnified in procedures that require the insertion of multiple instruments into an incision.

### SUMMARY

The present application fulfills a long felt need in the art for a compact device for uniportal endoscopic wireless viewing of a target site in a subject in need thereof.

One aspect of the present application relates to a wireless endoscopic viewing device. The device comprises a housing having a proximal end and a distal end; a circuit board having a processor, a transmitter for wirelessly communicating with at least one external receiver, and a power source, wherein the circuit board, transmitter, and power source are enclosed within the housing; a shaft having a proximal end and a distal end, wherein the proximal end of the shaft is attached to the distal end of the housing; and a camera attached to the distal end of the shaft, wherein the camera comprises a video flexible scope.

Another aspect of the present application relates to a system for wireless observation of a target tissue. The system comprises the wireless endoscopic viewing device and an external receiver for receiving images transmitted by the wireless endoscopic viewing device.

Still another aspect of the present application relates to a method for wireless observation of a target site in a subject in need thereof with the wireless endoscopic viewing device. The method comprises establishing an entry portal having access to the target site, inserting the distal end of the disposable cannula through the entry portal, advancing the cannula toward the target site; and imaging the target site with the camera.

### BRIEF DESCRIPTION OF THE DRAWINGS

The present application can be better understood by reference to the following drawings. The drawings are merely exemplary to illustrate certain features that may be used singularly or in any combination with other features and the present application should not be limited to the embodiments shown.

FIG. 1 shows an exemplary embodiment of the present application.

FIGS. 2A-C show top (2A), side (2B) and distal end (2C) perspective views of an exemplary embodiment of the present invention.

FIG. 3 shows an exemplary wireless viewing device of the present application.

FIG. 4 shows another exemplary wireless viewing device of the present application.

FIGS. 5A-D show an exemplary wireless viewing device of the present application having a fixed cannula.

FIGS. 6A-D show an exemplary wireless viewing device of the present application having a detachable cannula.

FIG. 7 shows another embodiment of the wireless viewing device of the present application.

FIG. 8 shows another embodiment of the wireless viewing device of the present application with a camera module (video flexible scope) and LED light source at the distal tip.

FIG. 9 shows an embodiment of a wireless receiver for images transmitted by the device.

### DETAILED DESCRIPTION

The following detailed description is presented to enable any person skilled in the art to make and use the object of this application. For purposes of explanation, specific nomenclature is set forth to provide a thorough understanding of the present application. However, it will be apparent to one skilled in the art that these specific details are not required to practice the subject of this application. Descriptions of specific applications are provided only as representative examples. The present application is not intended to be limited to the embodiments shown, but is to be accorded the widest possible scope consistent with the principles and features disclosed herein.

This description is intended to be read in connection with the accompanying drawings, which are to be considered part of the entire written description of this application. The drawing figures are not necessarily to scale and certain features of the application may be shown exaggerated in scale or in somewhat schematic form in the interest of clarity and conciseness. In the description, relative terms such as “front,” “back,” “up,” “down,” “top,” “bottom,” “upper,” “lower,” “distal,” and “proximal” as well as derivatives thereof, should be construed to refer to the orientation as then described or as shown in the drawing figure under discussion.

These relative terms are for convenience of description and normally are not intended to require a particular orientation. Terms concerning attachments, coupling and the like, such as “connected,” “mounted,” and “attached,” refer to a relationship wherein structures are secured or attached to one another either directly or indirectly through intervening structures, as well as both movable or rigid attachments or relationships, unless expressly described otherwise.

The term “trigger finger,” as used herein, also refers to “trigger digit,” “trigger thumb,” and “stenosing tendovaginitis.”

As used herein, the terms “horizontal” and “vertical,” and derivatives of those terms, are used in respect to their relationship to the plane defined by the slot in the cannula of the present application. “Vertical” refers to the plane that can, for example, pass through the slot of the cannula and bisect the cannula into two equal halves, while “horizontal” refers to a plane that is perpendicular to the vertical plane. The horizontal plane may be a level plane with respect to the length of the cannula or housing of the device, or may be at an angle to that level plane, allowing some upward or downward movement of elements moving along the horizontal plane with respect to the level plane.

As used herein, the term “subject” refers to an animal. In some embodiments, the animal is a mammal. In further embodiments, the mammal is a human.

As used herein, the term “practitioner” refers to one of skill in the art or any other user of the present device.

As used herein, the term “durable” refers to an object that can be used more than one time. A durable object can be sterilized by any means suitable for the nature of the object including, but not limited to, ethylene oxide (EtO), autoclaving, gamma irradiation, sterilizing wipes, sterilizing spray, or ultraviolet radiation.

The present application fulfills a long felt need in the art for a compact device for uniportal endoscopic wireless viewing of a target site in a subject in need thereof. The present device comprises integrated camera and lighting, allowing wireless real-time transmission of images to any viewing device, providing the advantage of allowing small clinics or individual practitioners to provide endoscopic services without the need to invest in costly equipment that can also be expensive to maintain.

The present application describes a compact viewing device for performing observations of bodily tissues or in conjunction with other devices to aid in the performance of procedures including, but not limited to, endoscopic surgical procedures. In some embodiments, a device of the present application comprises a rigid or flexible cannula that is attached to the distal end of a housing. In some further embodiments, the cannula is clear, being made of a transparent material. In some embodiments, the procedure can be a uniportal percutaneous endoscopic surgical procedure. In some embodiments, the viewing device of the present application is configured to be attachable to endoscopes, and laparoscopes. In some embodiments, the viewing device of the present application is configured to be attachable to existing surgical tools, such as clamps and wires, to add a camera to the existing tool.

In some embodiments, the cannula has an open proximal end where it is attached to the distal end of the housing. In some embodiments, the cannula comprises a closed distal end. In some further embodiments, the closed distal end of the cannula is upturned and comprises an edge for separating, but not cutting, tissues as the cannula is advanced from an entry portal towards a target tissue. In some embodiments, the cannula comprises a longitudinal slot that extends from the proximate end to the proximity of the distal end of the cannula. In some further embodiments, the distal end of the slot is contiguous with an open distal end of the cannula. In other further embodiments, the distal end of the slot is closed. In still other embodiments, the cannula comprises open proximate and distal ends, with the longitudinal surfaces of the cannula being closed.

The device comprises a sensor or camera for imaging a target area, a light source for illuminating the target area, a circuit board for controlling the functions of the device, and a transmitter/receiver/antenna/wire assembly for communicating between the device and a remote control panel or monitor.

In some embodiments, the sensor is an Omnivision® sensor and is provided ready for use in a compact camera module comprising included optics/lenses. In other embodiments, the sensor is an Omnivision® sensor and is provided “as is,” requiring additional optics/lenses. In still other embodiments, the sensor is a Medigus sensor.

In some embodiments, the sensor is less than 8 mm in width and is for medical/surgical applications. In some embodiments, the sensor is less than 7 mm in width and is for medical/surgical applications. In some embodiments, the sensor is less than 6 mm in width and is for medical/surgical applications. In some embodiments, the sensor is less than 5 mm in width and is for medical/surgical applications. In some embodiments, the sensor is less than 4 mm in width and is for medical/surgical applications. In some embodiments, the sensor has a width of 2-10 mm, 2-8 mm, 2-6 mm, 2-4 mm, 4-10 mm, 4-8 mm, 4-6 mm, 6-10 mm, 6-8 mm or 8-10 mm. In some embodiments, the sensor has a width of about 2, 3, 4, 5, 6, 7, 8, 9 or 10 mm.

In some embodiments, the light source is an LED light source in close proximity to the sensor. In other embodiments, a fiber or fiber bundle is connected the light source and transmits light from a light source remotely located in the viewing device to the sensor.

In some embodiments, the viewing device comprises an on-board video processing board/image processing unit. In some embodiments, the video processing board/image processing unit is an Omnivision® video processing board/image processing unit, such as, but not limited to, an OVMed-ISP image processing unit or an OV426. In other embodiments, the video processing board/image processing unit is a Medigus video processing board/image processing unit. In other embodiments, the video processing board/image processing unit is an A.M. Surgical video processing board/image processing unit.

In some embodiments, the transmitter is a wireless transmitter. In some embodiments, the wireless transmitter is a microchip transmitter. In some embodiments, the wireless transmitter comprises an Amimon transceiver chip. In some embodiments, the wireless transmitter and at least one external receiver communicate via wireless local area networking based on the IEEE 802.11 standards (WiFi). In other embodiments, communication is via WIHD. In other embodiments, communication is via WirelessHD (such as Ultragig). In other embodiments, communication is via WiGig. In some embodiments, communication is via radio. In other embodiments, communication is via short-wavelength radio transmissions, for example in the ISM band from 2400-2480 MHz (IEEE 802.15.1, or Bluetooth®). In some embodiments, communication is via radio frequency (RF) communication signals (e.g., FM radio signal). In other embodiments, communication is via microwave or infrared (IR) communication signals from the wireless sensor. In other embodiments, communication is via near-field communication (NFC) signals between the internal control board having transmitter and receiver functions for wirelessly communicating and the at least one external receiver and transmitter. In certain embodiments, the wireless transmitter may utilize satellite communication. In various embodiments, the wireless transmitter utilizes wireless sensor networks such as ZigBee®, EnOcean®, TransferJet®, Ultra-wideband; or short-range point-to-point communication such as radio frequency identification (RFID). In some embodiments, communication is via digital communication. In other embodiments, communication is via analog communication.

In some embodiments, the viewing device transmits video data via wire or cable to a receiver. In some embodiments, the wire-connected receiver preforms the function of processing the video sensor input and delivers the data to a monitor or other visual medium. In some embodiments, the wire or cable is USB, mini-USB, micro-USB, USB-C or Lightning. In some embodiments, the wire-connected receiver is held or worn by the practitioner.

In some embodiments, the viewing device is capable of transmission to a receiver via a combination of any of the above methods.

In some embodiments, the communication between the transmitter and the receiver is a closed, paired system. In other embodiments, signal from the transmitter can be received by multiple receivers.

In some embodiments, the external receiver is interfaced with a computer terminal or video monitor. In some further embodiments, the computer terminal is a notebook computer. In other embodiments, the external receiver is a tablet or smart phone. In some further embodiments, the computer, tablet or smart phone comprises an application (app) that communicates with the viewing device. In still further embodiments, the app that communicates with the viewing device is a dedicated app. In some embodiments, the viewing device is provided with a unique identifier that can be entered into/associated with the app for dedicated communication between an individual viewing device and the app. In some embodiments, the app is capable of recording the transmission from the viewing device as a video or individual pictures/screen captures. In further embodiments, the recordings can be saved into an archive, such as a medical record of the subject. In particular embodiments, the app is capable of remotely controlling functions of the viewing device. For example, functions that could be controlled remotely include, but are not limited to, camera focus, camera optical zoom, camera digital zoom, camera field of view, camera angle, camera rotation, light on/off, and light intensity.

In some embodiments, the receiver is a dongle connected to a monitor. In some embodiments, the receiver is connected via wire or cable to one or more displays. In other embodiments, the receiver relays data wirelessly to one or more displays.

The monitor can be any type of monitor, including but not limited to a video monitor, audio monitor, wavelength monitor, etc. One embodiment, the monitor is a video monitor. The housing further contains a power source, such as a battery. The preassembled nature of the device also provides convenience for the practitioner in that the cannula, camera and tools are available in a single package that requires no further assembly and can be used easily in an office setting without the need for some traditional endoscopic equipment that may be too expensive or cumbersome to use outside of a hospital. Additionally, the present device also can be easily transported and used in remote settings, such as by emergency medical personnel, first responders or military medical personnel.

In some embodiments, the device is sterilized before use or before delivery. In some embodiments, the entire device is disposable. In other embodiments, the entire device is durable. In still other embodiments, the cannula element is disposable while the remainder of the device is durable. In some embodiments, durable portions of the device can be re-sterilized before or after use. Methods of sterilization include, but are not limited to, ethylene oxide, autoclaving, gamma irradiation, sterilizing wipes, sterilizing spray, and/or ultraviolet radiation. In some embodiments, the device is fully reusable and can be sterilized by ethylene oxide, autoclaving, gamma irradiation, sterilizing wipes, sterilizing spray and/or ultraviolet radiation.

In some embodiments, the device can be used for any general or surgical application.

In some embodiments, the device can be used for a uniportal endoscopic viewing and/or surgical procedure. In other embodiments, the device can be used for an endoscopic, laparoscopic, or thoracoscopic viewing and/or surgical procedure. As used herein, “laparoscopic” and “thoracoscopic” procedures fall within the scope of “endoscopic” procedures.

In some embodiments, the cannula of the device is adapted for use in orthopedic procedures. The device may be used as an endoscope or laparoscope. In some embodiments, the device can be used with, and enhance the function or utility of other devices, such as clamps or wires, by adding camera functionality.

In some embodiments, the cannula of the device is insertable into another device. For example, the device can be used in the place of a traditional endoscope in conjunction with a compact endoscopic surgical device, such as STRATOS® (A.M. Surgical, Inc., Smithtown, N.Y.) or other devices. In some embodiments, the components of the device, can be incorporated into another device, such as STRATOS®, to create an endoscopic surgical device with an embedded camera.

Endoscopic surgical procedures that can be performed with a device of the present application include, but are not limited to, carpal tunnel release, Guyon's canal (or tunnel) release, cubital tunnel release, plantar fascia release, lateral release for patella realignment, release of radial tunnel, release of pronatar teres, release of trigger finger, release of lacertus fibrosus, tendon release, release of the extensor tendons for lateral epicondylitis, release of medial epicondylitis, release of the posterior and other compartments of the leg, forearm fascia release for fascial compartment syndrome, release of fascial compartments in the upper or lower extremities, relieving the compression of a nerve by a ligament pulley or tunnel, and releasing the travel of a ligament or tendon through a pulley or tunnel. Procedures that can be performed with a cannula or device of the present application include endoscopic surgical procedures on the spine, such as discectomy for the treatment of degenerative disc disease, herniated discs, bulging discs, pinched nerves or sciatica. Procedures that can be performed with a cannula or device of the present application also include procedures on cranial and facial tissues, as well as fasciotomy release throughout the body. The cannula or device of the present application can be used for blood vessel, including vein or artery, harvesting throughout the body, for example to provide blood vessel graft material in conjunction with a coronary bypass procedure or for a reconstructive surgical procedure. Procedures that can be performed with a cannula or device of the present application also include endoscopic procedures on the wrist and hand, including the palmar and dorsal sides of the hand. Endoscopic procedures that can be performed with a cannula or device of the present application on the hand also include the digits, including the thumb, index finger, middle finger, ring finger and little (pinky) finger. Other examples of endoscopic procedures that can be performed with a device of the present application include, but are not limited to, observation of internal tissues or injuries, cauterization of vessels, harvesting of tissues for ex vivo growth; obtaining biopsies; spinal surgery; endonasal surgery; mucosal resection; removal of parasites, cysts or tumors, and foreign body retrieval. Still other examples of endoscopic surgery that can be performed with the device include, but are not limited to, procedures on or within bone, in or around joints or the tendons associated with those joints, as well as any tissue, area or cavity of the body of a subject. In some embodiments, endoscopic surgical procedures, including, but not limited to, carpal tunnel release, can be performed using a viewing device of the present application in the place of a traditional endoscope in conjunction with a compact endoscopic surgical device, such as STRATOS® (A.M. Surgical, Inc., Smithtown, N.Y.) or other devices.

In some embodiments, the present device can be used in the head of a subject. Exemplary procedures in the head include, but are not limited to, nasal surgery, endoscopic sinus surgery, endoscopic pituitary surgery, cranial surgery, endoscopic ear surgery, throat surgery, endodontic surgery and tonsils.

In some embodiments, the present device can be used in the neck of a subject. Exemplary procedures in the neck include, but are not limited to, laryngoscopic surgery, vocal cord surgery, esophageal surgery, thyroid surgery, carotid artery surgery, and brachial plexus surgery.

In some embodiments, the present device can be used in the chest of a subject. Exemplary procedures in the chest include, but are not limited to, endoscopic mediastinal surgery, thoracic surgery, heart surgery, esophageal surgery, and upper gastrointestinal (GI) scoping.

In some embodiments, the present device can be used in a procedure of a finger, hand, foot of a subject.

In some other embodiments, the present device can be used in the abdomen of a subject. Exemplary procedures in the abdomen include, but are not limited to, diagnostic laparoscopy, laparoscopic gastric surgery, laparoscopic liver surgery, laparoscopic pancreatic surgery, laparoscopic nephrectomy and kidney surgery, laparoscopic intestinal surgery, laparoscopic oophorectomy, laparoscopic hysterectomy, laparoscopic urinary bladder surgery, laparoscopic prostate surgery, laparoscopic aortic surgery, laparoscopic appendectomy, laparoscopic colon surgery, endoscopic hysterotomy, endoscopic fetal surgery, endoscopic hernia repair, and endoscopic splenectomy.

In some embodiments, the present device can be used in an upper extremity of a subject. Exemplary procedures in an upper extremity include, but are not limited to, ECTR, ECUTR, endoscopic pronator teres release, forearm fascial compartment release, endoscopic repair of biceps tendon, endoscopic release of lateral and medial epicondylitis, endoscopic release of radial tunnel syndrome, endoscopic surgery of the brachial plexus, endoscopic harvesting of nerve graft, endoscopy and surgery of wrist, endoscopy of elbow, endoscopy and surgery of the carpometacarpal (CMC) joint, endoscopy and surgery of shoulder, endoscopy and surgery of acromioclavicular (AC) joint.

In some embodiments, the present device can be used in a lower extremity of a subject. Exemplary procedures in an lower extremity include, but are not limited to, femoral artery surgery, fascia lata release, knee lateral release, endoscopic peroneal nerve release, endoscopic leg fascial compartment release, endoscopic release of gastrocnemius, endoscopic tarsal tunnel release, endoscopic release of Morton's neuroma, endoscopic release of the plantar fascia, endoscopy of hip, knee and ankle, subtalar joint, and endoscopic harvesting of nerve and tendon graft.

Endoscopic surgical procedures that can be performed with a device of the present application, such as, but not limited to, a ligament or fascia release procedure, can be performed by approaching the target tissue through an incision or body opening on either the proximate or distal side of the target tissue.

In some embodiments, a device of the present application can be used for plastic surgery. A device of the present application is useful for tissue remodeling or the excision of tissue segments, including necrotic tissue.

A device of the present application is lightweight, compact and can be manipulated with a single hand. The weight of the device is less than about one pound, allowing the device to be easily carried within a pocket, backpack, satchel or case. In some embodiments, the device weighs less than about 15, 14, 13, 12, 11, 10, 9, 8, 7, 6, 5, 4 or 3 ounces.

The housing of the device can be generally rectangular or oval in shape. The housing can also serve as a grip or handle for the device. All on-board components of the device are housed within a single housing, or within the housing and the cannula. In general, the housing of the device can be easily held within the palm of one hand and manipulated by that one hand. In some embodiments, the overall dimensions of the housing (with the longest dimension being measured from the distal end to the proximal end of the housing) are less than about 7 inches in length, 2 inches in width and 2 inches in thickness (7×2×2). In further embodiments, the overall dimensions of the housing are less than about 6×2×1. In still further embodiments, the overall dimensions of the housing are less than about 5×1.5×1. In even further embodiments, the overall dimensions of the housing are less than about 5×1.5×0.5. The outer surface of the housing may be textured, grooved, indented or shaped to facilitate gripping by a hand or by another device.

The cannula of the device is attached at its proximal end to the distal end of the housing. The cannula can be either permanently attached to the distal end, or may be detachable/replaceable by any suitable means including, but not limited to, a luer-lock type system, cam lock, snap-fit or threaded to screw into or onto the distal end of the housing. The cannula can be opaque, translucent or transparent. The cannula can be flexible or rigid. In some embodiments, the cannula is made of polycarbonate. In some embodiments a cannula of the present device is about 12 inches in length. In some embodiments, a cannula of the present device is about 11, 10, 9, 8, 7, 6, 5, 4, 3 or 2 inches in length. In some embodiments, the cannula is a rigid, transparent, cylinder-shaped tube with an open slot extending from its proximal end or the proximity of its proximal end to its distal end or the proximity of its distal end.

In some embodiments, one or more detachable cannulas may be supplied with a housing as part of a kit. Each cannula supplied with the housing may be designed or suited for a particular need for the examination and/or treatment of a subject. For example, in a kit with multiple cannulas supplied, the practitioner can select a cannula that is best suited for use with the immediate treatment/examination need of the subject. The cannulas can be interchanged during the treatment of that subject based upon changing needs by the practitioner for the treatment/examination of that subject.

The compact size and light weight of the device reduces the amount of fatigue experienced by the practitioner operating the device versus larger, heavier devices.

The device can be supplied as a single-use, disposable device that is pre-sterilized and sealed within packaging that keeps the device sterile until opened. In some embodiments, the device is fully disposable and comes pre-sterilized via ethylene oxide (EtO). The device can be supplied as part of a kit that includes additional instruments useful with the device such as, but not limited to, scalpel, elevator, dilator, bandages, tape, needles and sutures.

The device can be used in a clinical setting. The clinical setting can be a hospital, emergency clinic, outpatient clinic, or office, for example. The device can also be used outside the clinical setting, such as, but not limited to, in an emergency situation. The device of the present application can be used by various practitioners including, but not limited to, a physician, surgeon, nurse, nurse practitioner, first responder, paramedic, emergency medical technician, medic, corpsman, technician or caregiver.

One aspect of the present application relates to a reusable wireless endoscopic viewing device. The device comprises a durable housing having a proximal end and a distal end; a circuit board having a processor, a transmitter for wirelessly communicating with at least one external receiver, and a power source, wherein the circuit board, transmitter, and power source are enclosed within the housing; wherein wires connected at their proximal ends to the circuit boards extend out through a distal opening in the housing, connecting at their distal ends to a camera or a light source. The device further comprises a disposable cannula having an open proximal end and a closed distal end, wherein the proximal end of the cannula releasably attaches to the distal end of the housing; wherein the open proximal end of the cannula releasably attaches to the housing such that the camera, light source and portion of the wires outside the housing are encased within the cannula.

In some embodiments, the device can be handheld or held by another device.

In some embodiments, the device can be used on any target tissue, bone, joint or target area of the body of a subject.

In some embodiments, the transmitter is a microchip transmitter.

In some embodiments, the lens of the camera has a diameter of less than 5 mm. In some further embodiments, the camera is a NANEYE® camera.

In some embodiments, the light source is an LED light source.

In some embodiments, the cannula is rigid.

In some embodiments, the cannula is flexible.

In some embodiments, the cannula is clear.

In some embodiments, the housing further comprises a slot for the insertion of a memory device. In some further embodiments, the memory device is selected from the group consisting of an SD card, a micro-SD card, a USB device and a flash drive.

Another aspect of the present application relates to a system for wireless observation of a target tissue, comprising a reusable wireless endoscopic viewing device. The device comprises a durable housing having a proximal end and a distal end; a circuit board having a processor, a transmitter for wirelessly communicating with at least one external receiver, and a power source, wherein the circuit board, transmitter, and power source are enclosed within the housing; wherein wires connected at their proximal ends to the circuit boards extend out through a distal opening in the housing, connecting at their distal ends to a camera or a light source. The device further comprises a disposable cannula having an open proximal end and a closed distal end, wherein the proximal end of the cannula releasably attaches to the distal end of the housing; wherein the open proximal end of the cannula releasably attaches to the housing such that the camera, light source and portion of the wires outside the housing are encased within the cannula. The system further comprises an external receiver/transmitter for visualizing images transmitted by the device.

In some embodiments, the external wireless receiver/transmitter is selected from the group consisting of video monitors, computer terminals comprising a monitor, smart phones and tablet computers.

In some embodiments, function of the device is controlled with a dedicated application (app) installed or resident on the external wireless receiver/transmitter.

Another aspect of the present application relates to a method for wireless observation of a target site in a subject in need thereof. The method comprises the steps of establishing an entry portal having access to the target site; inserting the distal end of a disposable cannula of a reusable wireless viewing device through the entry portal, wherein the device comprises a durable housing having a proximal end and a distal end; a circuit board having a processor, a transmitter for wirelessly communicating with at least one external receiver, and a power source, wherein the circuit board, transmitter, and power source are enclosed within the housing; wherein wires connected at their proximal ends to the circuit boards extend out through a distal opening in the housing, connecting at their distal ends to a camera or a light source. The device further comprises a disposable cannula having an open proximal end and a closed distal end, wherein the proximal end of the cannula releasably attaches to the distal end of the housing; wherein the open proximal end of the cannula releasably attaches to the housing such that the camera, light source and portion of the wires outside the housing are encased within the cannula; advancing the cannula toward the target site; and imaging the target site with the camera.

In some embodiments, the target site is a joint.

In some embodiments, the target site is a ligament, tendon or pulley.

In some embodiments, the target site is fascia.

In some embodiments, the target site is a blood vessel.

Another aspect of the present application relates to a kit for wireless observation of a target site in a subject. The kit contains a reusable wireless viewing device comprising a durable housing having a proximal end and a distal end; a circuit board having a processor, a transmitter for wirelessly communicating with at least one external receiver, and a power source, wherein the circuit board, transmitter, and power source are enclosed within the housing; wherein wires connected at their proximal ends to the circuit boards extend out through a distal opening in the housing, connecting at their distal ends to a camera or a light source; at least one disposable cannula having an open proximal end and a closed distal end, wherein the proximal end of the cannula releasably attaches to the distal end of the housing; wherein the open proximal end of the cannula releasably attaches to the housing such that the camera, light source and portion of the wires outside the housing are encased within the cannula.

In some embodiments, the kit comprises more than one cannula.

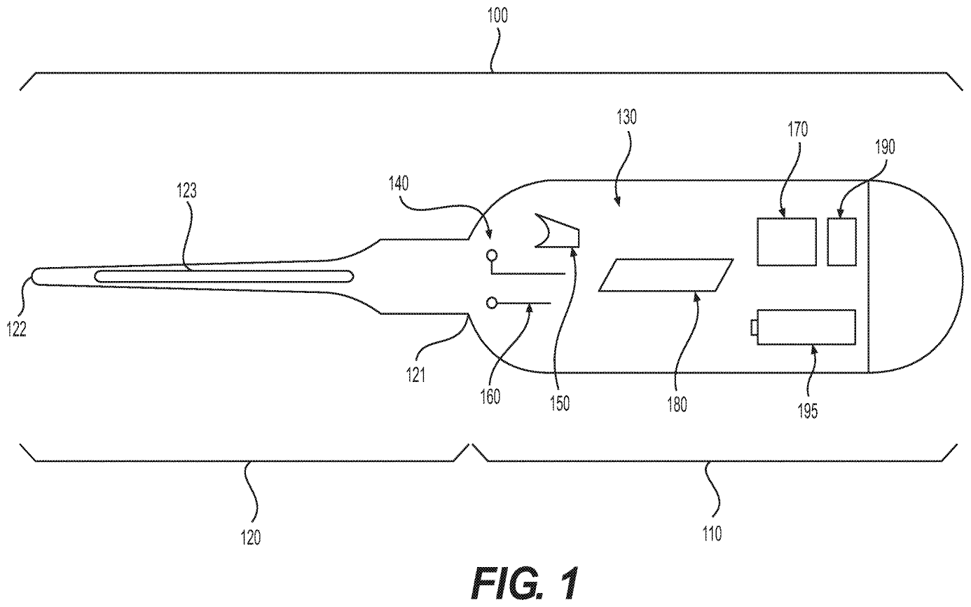

FIG. 1 shows one exemplary embodiment of the device 100 of the present application. The device generally comprises a housing 110 and a cannula 120. The cannula 120 comprises an open central lumen, a proximal end 121 and a distal end 122, wherein the proximal end 121 of the cannula 120 is attached to the distal end of the housing 110.

In some embodiments, the cannula 120 is composed of a clear material. In further embodiments, the clear material is polycarbonate. In some embodiments, the cannula is marked with gradations showing how far the cannula 120 had been inserted through an entry portal.

The cannula 120 of the present device 100 comprises a slot 123 in its upper surface, wherein said slot 123 is contiguous with the open central lumen. In some embodiments, said upper surface is flattened, in other embodiments, said upper surface is rounded. In some embodiments, the slot 123 extends from the proximal end 121 to the proximity of the distal end 122 of the cannula 120. In other embodiments, the slot extends from a point located between the proximal end 121 and distal end 122 to the proximity of the distal end 122. As used herein, “the proximity of the distal end” has the meaning of the slot 123 ending prior to actually joining the distal end 122, i.e., having at least a minimal bridge of material crossing between the distal end of the slot 123 and the distal end 122 of the cannula 120 to prevent over-advancement of a deployed tool through the distal end 122 of the cannula 120.

In other embodiments, the distal end of the slot 123 is contiguous with an open distal end 122 of the cannula 120.

In some embodiments, the distal end 122 of the cannula 120 is closed, as an obturator. In some embodiments, said distal end 122 is pointed. In other embodiments, the distal end 122 comprises a leading edge that is turned upwards, allowing the cannula to separate and form a passage from the entry portal through/between/under/over body tissues to and/or past a target tissue. In some further embodiments, the edge can be flattened.

In some embodiments, the body of the cannula 120 is laterally expandable in order to spread tissue as a passage is made by the cannula 120, obviating the need for inserting a separate instrument through the entry portal to spread tissue.

Also as shown in FIG. 1, the camera 130 and surgical tools are contained within the housing 110 of the device 100 prior to deployment into the cannula 120.

The device 100 comprises a camera 130 that is small enough to deploy into the central lumen of the cannula 120. The camera 130 is generally a high resolution camera, but is at least of sufficient resolution for imaging with sufficient clarity to distinguish different bodily tissues from one another and to image a target tissue with sufficient clarity in order to observe the performance of a surgical procedure on the target tissue. In some embodiments, the camera 130 can be focused. The camera 130 can be advanced into the cannula 120 independently of any surgical tools in order to image/observe bodily tissues or target tissue surrounding the cannula 120 or through the slot 123 before, after or in lieu of a surgical procedure. Having an integral camera 130 within the device, eliminates the need to insert a separate endoscopic camera into the device or an entry portal, thereby eliminating the need for another separate element in the procedure.

The camera 130 can also be advanced into the cannula 120 in association with the probe 140, blade 150, cautery 160 or other suitable surgical tool. In general, the camera comprises within its field of view any portion of the probe 140, blade 150, cautery 160 or other suitable surgical tool that is in contact with, or performing a desired surgical procedure on, a target or bodily tissue. In some embodiments, the camera is a NANEYE® camera. In other embodiments, the camera 130 has a resolution of at least 100×100 pixels. In a further embodiment, the camera 130 has a resolution of at least 150×150 pixels. In a still further embodiment, the camera 130 has a resolution of at least 200×200 pixels. In an even further embodiment, the camera 130 has a resolution of at least 250×250 pixels. In some embodiments, there is a separate camera 130 independently associated with each tool of the device 100.

In another embodiment, the camera 130 remains in a fixed position within the housing 110. In a further embodiment, the camera 130 comprises an image transmitting optical fiber, which is attached at its proximal end to the camera. In some still further embodiments, the distal end of the image transmitting optical fiber is movable and moves into the cannula 120 independently or with tools of the device 100. In other still further embodiments, the distal end of the image transmitting optical fiber is in a fixed position in the proximity of the distal end 122 of the cannula, such that from the fixed position of the image transmitting optical fiber the camera 130 can observe and image the surgical procedure.

In still another embodiment, the device 100 comprises a combination of at least one movable camera 130 and at least one fixed position camera 130 as described above. In some embodiments, a camera 130 of the present device 100 comprises a camera body and a lens assembly that is attached to the camera body via an image transmitting optical fiber.

Still referring to FIG. 1, in some embodiments, the device comprises a probe element 140. The probe 140 can be advanced into the cannula 120 and protrudes vertically through the slot 123 in order to, for example, move tissues above the slot 123, provide a reference point for imaging, determine the edges of the target tissue or remove synovium from the target tissue.

Also referring to FIG. 1, the device 100 comprises a blade 150 for performing surgical procedures on a target tissue. The blade 150 can be advanced into the cannula 120 and protrudes vertically through the slot 123 in order to divide a target tissue. In some embodiments, the blade 150 comprises at least one cutting surface on its distal side and division of the target tissue is performed by moving the blade through the slot 123 in a proximal 121 to distal 122 direction. In other embodiments, the blade 150 comprises at least one cutting surface on its proximal side and division of the target tissue is performed by moving the blade through the slot 123 in a distal 122 to proximal 121 direction.

FIG. 1 also shows an embodiment of the device 100 comprising a cautery element 160. The cautery 160 can be advanced into the cannula 120 and protrudes vertically through the slot 123 in order to cauterize a target tissue. In some embodiments, the target tissue was previously divided with the blade 150 of the device 100 during the same surgical procedure. In another embodiment, the target tissue was previously divided by a blade in an earlier surgical procedure. In yet another embodiment, the target tissue was in need of cauterizing due to an earlier injury or other outstanding medical condition.

In some embodiments, the device 100 further comprises a light source 170 contained within the housing 110. The light source 170 provides illumination for the camera 130 in order to allow visualization of bodily or target tissues through the cannula 120 or slot 123. In some embodiments, the position of the light source 170 is fixed within the housing 110 of the device 100. In other embodiments, the light source 170 is associated with the camera 130 and travels with the camera 130, either into the cannula 120, or staying within the housing 110, but moving closer to the proximal end 121 of the cannula 120 as the camera 130 is advanced toward the distal end 122 of the cannula 120. In still other embodiments, the light source 170 comprises a main body whose position is fixed within the housing and is attached to the proximal end of a light transmitting fiber, the distal end of which provides light for the camera 130. In some further embodiments, the distal end of the light transmitting fiber moves in concert with the camera 130. In other further embodiments, the distal end of the light transmitting fiber remains in a fixed position within the cannula 120, for example, in the proximity of the distal end 122 of the cannula. In some embodiments, the light source 170 is a semiconductor light source. In some embodiments, the light source 170 is a light emitting diode (LED) light source. In some embodiments, the device 100 comprises a plurality of light sources 170 as described above that can be in a fixed position and/or moveable.

Still in FIG. 1, in some embodiments, the device 100 comprises a circuit board 180 for processing imagery obtained by the camera 130. Said imagery is transmitted to a remote control or video display via a wireless antenna 190 contained within the housing 110 of the device 100. In some instances, the circuit board receives instructions from the remote control via the wireless antenna 190. In some embodiments, the movement of the camera 130 and the tools of the device 100 are controlled remotely via instruction transmitted to the circuit board 180. In other embodiments, the housing 110 of the device 100 comprises manual control for selecting tools and/or advancing/withdrawing tools or the camera 130 into/from the cannula 120.

Also depicted in FIG. 1, the device 100 further comprises an integral power source 195 to provide energy for the camera 130, light source 170, circuit board 180 and any mechanical functions within the device 100. In some embodiments, the power source 195 is a battery. In further embodiments, the battery is a lithium battery. In some embodiments, the power source 195 is installed within the device 100 upon manufacture, or prior to provision to a practitioner. In other embodiments, the power source 195 is provided separately from the device 100 and installed into the device 100 prior to the use of the device 100 in a surgical procedure. In some embodiments, the power source 195 is removable from the device 100 for separate disposal.

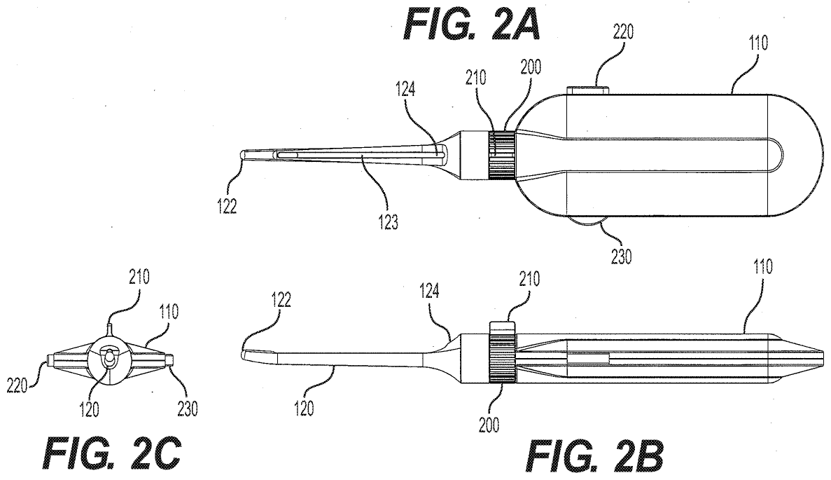

FIG. 2A shows a top view of an embodiment of the device, showing a cannula 120 attached at its proximal end to the distal end of a housing 110. In this embodiment, the cannula 120 comprises a longitudinal slot 123 that extends longitudinally from a proximal end 124 to near the distal end 122 of the cannula 120. In some embodiments, the proximal end 124 of the slot is raised to better allow tools to enter the slot without scraping against the proximal end 124. In some embodiments, at the junction of the housing 110 and the cannula 120, the device comprises a connecting ring 200 that attaches the cannula 120 to the housing 110. In some embodiments, the connecting ring 200 comprises a paddle 210 for ease of manipulation, such as when the device is being held and controlled within one hand. In some embodiments, the connecting ring allows for the interchangeable attachment of different types of cannulas 120 to a given housing 110, dependent upon the procedure to be performed with the device. In certain embodiments, selected tools, such as the probe 140 shown in FIG. 1, are moved into and out of the cannula 120. In some embodiments, the housing 110 further comprises a rocker switch 220 that is used to move the selected tools, such as the probe 140, the blade 150, the cautery 160 or other suitable surgical tools within the housing into and out of the cannula 120. In other embodiments, the movement of tools such as the probe 140, the blade 150, the cautery 160 or other suitable surgical tools into and out of the cannula 120 is controlled electronically and/or remotely. In some embodiments, the housing 110 also comprises a switch 230 for turning the light on and off.

FIG. 2B is a side view of the embodiment of the device shown in FIG. 2A, showing the housing 110, cannula 120, and the intervening connecting ring 200 with paddle 210. In some embodiments, the distal end 122 of the cannula 120 is closed and turned upwards, serving as an integral obturator.

FIG. 2C is a distal end view of the embodiment of the device shown in FIG. 2A, showing the relative positions of the cannula 120, housing 110, paddle 210, slider knob 220 and switch 230.

Another aspect of the present application relates to a wireless viewing device for observation of bodily tissues. The wireless viewing device comprises a housing having a proximal end and a distal end; a wand having a proximal end and a distal end, wherein the proximal end of the wand adjoins the distal end of the housing; a camera with a lens; a light source; a control board having transmitter functions for wirelessly communicating with at least one external receiver; and a power source, wherein the control board and power source are enclosed by the housing and the camera with a lens and the light source are located at or proximate to the distal end of the wand. In some embodiments, the wireless viewing device is insertable into a cannula.

As with other embodiments of the present application, the wireless viewing device of this embodiment can be used on any target tissue, bone, joint or target area of the body of a subject as set forth above. In some embodiments, the wireless viewing device of this embodiment can be used for minimally-invasive observation of an internal bodily target location. The wireless viewing device can be used, for example, for pre-treatment, or pre-operative, observation or diagnosis; post-treatment, or post-operative, observation or follow-up; or general observation of a target location in need thereof without further treatment or operative intervention. In some embodiments, the wireless viewing device of this embodiment can be used for monitoring of the progress or regression of a condition or target tissue, such as a surgical repair of a target tissue or the growth/regression of a neoplasm or tumor. The device comprises a housing having a proximal end and a distal end; a wand having a proximal end and a distal end, wherein the proximal end of the wand adjoins the distal end of the housing; a camera with a lens; a light source; a control board having transmitter functions for wirelessly communicating with at least one external receiver; and a power source, wherein the control board and power source are enclosed by the housing and the camera with a lens and the light source are located at or proximate to the distal end of the wand. In some embodiments, the wireless viewing device is insertable into a cannula.

A wireless viewing device as embodied by the present application has an advantage for the practitioner and the subject in that it can be provided sterile as a single use and disposable device, without the need to be physically attached to monitoring or viewing equipment, thereby eliminating the possibility of transmitting infectious agents between subjects, which is a risk with reusable instruments that must be sterilized between uses. Additionally, the wireless viewing device can be used with a variety of devices that traverse an entry portal into the body of a subject for observation of a target tissue. Such devices that traverse an entry portal include, but are not limited to, a cannula, anoscope, port or any other suitable tubular entry device. In some embodiments, both the wireless viewing device and the device that traverses an entry portal are single use and disposable, further enhancing the elimination of the possibility of transmitting infectious agents between subjects.

The wireless viewing device described herein is completely self-contained within the singular housing and wand. The present device eliminates the need for connectors or attachments such as external pods comprising additional power sources, light sources, transmitters & receivers or imaging equipment. This elimination of the need for additional components simplifies the use for the practitioner, as well as enhances the sterility of the device.

A wireless viewing device of the present application is lightweight, compact and can be manipulated with a single hand. The weight of the device is less than about one pound, allowing the device to be easily carried within a pocket, backpack, satchel or case. In some embodiments, the device weighs less than about 15, 14, 13, 12, 11, 10, 9, 8, 7, 6, 5, 4 or 3 ounces.

The housing of the wireless viewing device can be generally rectangular or oval in shape. The housing can also serve as a grip or handle for the device. All on-board components of the device are housed within a single housing, or within the housing and the wand. In general, the housing of the device can be easily held within the palm of one hand and manipulated by that one hand. In some embodiments, the overall dimensions of the housing (with the longest dimension being measured from the distal end to the proximal end of the housing) are less than about 7 inches in length, 2 inches in width and 2 inches in thickness (7×2×2). In further embodiments, the overall dimensions of the housing are less than about 6×2×1. In still further embodiments, the overall dimensions of the housing are less than about 5×1.5×1. In even further embodiments, the overall dimensions of the housing are less than about 5×1.5×0.5. The outer surface of the housing may be textured, grooved, indented or shaped to facilitate gripping by a hand or by another device.

The cannula of the wireless viewing device is attached at its proximal end to the distal end of the housing. The wand can be either permanently attached to the distal end, or may be detachable/replaceable by any suitable means including, but not limited to, a luer-lock type system, cam lock, snap-fit or threaded to screw into or onto the distal end of the housing. The wand can be opaque, translucent or transparent. The wand can be flexible or rigid. In some embodiments, the wand is made of polycarbonate. In some embodiments a wand of the present device is about 12 inches in length. In some embodiments, a wand of the present device is about 11, 10, 9, 8, 7, 6, 5, 4, 3 or 2 inches in length.

In some embodiments, one or more detachable wands may be supplied with a housing as part of a kit. Each wand supplied with the housing may be designed or suited for a particular need for the examination and/or treatment of a subject. For example, in a kit with multiple wands supplied, the practitioner can select a wand that is best suited for use with the immediate treatment/examination need of the subject. The wands can be interchanged during the treatment of that subject based upon changing needs by the practitioner for the treatment/examination of that subject.

The wireless viewing device can be supplied as a single-use, disposable device that is pre-sterilized and sealed within packaging that keeps the device sterile until opened. The device can be supplied as part of a kit that includes additional instruments useful with the device such as, but not limited to, scalpel, elevator, dilator, bandages, tape, needles and sutures.

The wireless viewing device can be used in a clinical setting. The clinical setting can be a hospital, emergency clinic, outpatient clinic, or office, for example. The device can also be used outside the clinical setting, such as, but not limited to, in an emergency situation. The device of the present application can be used by various practitioners including, but not limited to, a physician, surgeon, nurse, nurse practitioner, first responder, paramedic, emergency medical technician, medic, corpsman, technician or caregiver.

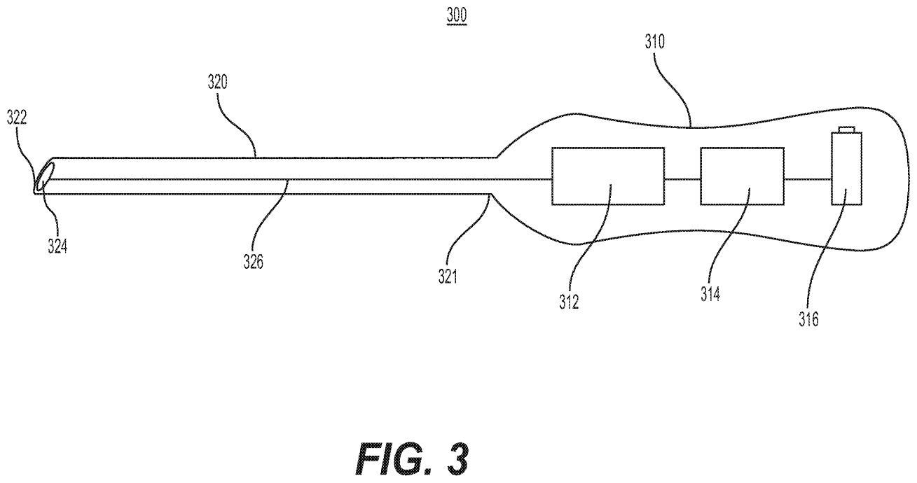

FIG. 3 illustrates an exemplary wireless viewing device 300 of the present application. The wireless viewing device comprises a housing 310 that also serves as a handle for the device and a wand 320, the proximal end of which is attached to the distal end of the housing 310. In some embodiments, the device can be handheld. In some further embodiments, the housing is formed in a shape to facilitate gripping with a single hand. In some embodiments, the device can be manually controlled. In other embodiments, the device can be remotely controlled. In some embodiments, the device can be held by another device. In some embodiments, the device could be attached to, held, by, inserted into or under the control of a robotic device or tool. In some embodiments, a light source 312 is contained within the housing 310. In some embodiments, the light source 312 is an LED light source. In some embodiments, the light source 312 shines through an opening in the distal end of the housing that is contiguous with the central lumen of the wand 320 to provide illumination for the camera 324. In other embodiments, the light source 312 illuminates a fiber optic 326 that extends into the wand 320. In still other embodiments, the light source 312 is located at the distal end of the wand 320.

Still referring to FIG. 3, the housing 310 further contains an internal control board having transmitter functions 314 that is capable of, for example, sending video images obtained by the wireless viewing device 300 to least one external receiver. In some embodiments, the device comprises an information display provided in the housing 310 to display frequency information of a transmission frequency of the wireless transmitter. In some embodiments, the internal control board has receiver functions, such that functions of the wireless viewing device can be controlled remotely. For example, functions that could be controlled remotely include, but are not limited to, camera focus, camera optical zoom, camera digital zoom, camera field of view, camera angle, camera rotation, light on/off, and light intensity.

In some embodiments, the internal control board having transmitter functions 314 for wirelessly communicating and the at least one external receiver communicate via radio frequency communication signals (e.g., FM radio signal). In other embodiments, communication is via microwave or infrared (IR) communication signals from the wireless sensor. In still other embodiments, communication is via short-wavelength radio transmissions, for example in the ISM band from 2400-2480 MHz (IEEE 802.15.1, or Bluetooth®). In even other embodiments, communication is via near-field communication (NFC) signals between the internal control board having transmitter and receiver functions for wirelessly communicating and the at least one external receiver and transmitter.

In some embodiments, the external receiver is interfaced with a computer terminal or video monitor. In some further embodiments, the computer terminal is a notebook computer. In other embodiments, the external receiver is a tablet or smart phone. In some further embodiments, the tablet or smart phone comprises an application (app) that communicates with the wireless viewing device 300. In still further embodiments, the app that communicates with the wireless viewing device 300 is a dedicated app. In some embodiments, the wireless viewing device 300 is provided with a unique identifier that can be entered into/associated with the app for dedicated communication between an individual wireless viewing device 300 and the app. In some embodiments, the app is capable of recording the transmission from the wireless viewing device as a video or individual pictures/screen captures. In further embodiments, the recordings can be saved into an archive, such as a medical record of the subject. In particular embodiments, the app is capable of remotely controlling functions of the wireless viewing device. For example, functions that could be controlled remotely include, but are not limited to, camera focus, camera optical zoom, camera digital zoom, camera field of view, camera angle, camera rotation, light on/off, and light intensity.

In other embodiments, the housing 310 further comprises a video display showing real time images from a camera 324 of the device.

In some embodiments, the wireless viewing device 300 comprises non-volatile memory for storing images or information from a performed procedure, wherein said images or information can be retrieved from the device. In some embodiments, the non-volatile memory is an RFID tag. In other embodiments, the non-volatile memory is a micro-SD card.

As shown in FIG. 3, in some embodiments, the housing further comprises a power source 316. In some embodiments, the power source 316 is a battery. In further embodiments, the battery is a lithium battery. In some embodiments, the power source 316 is installed within the wireless viewing device 300 upon manufacture, or prior to provision to a practitioner. In other embodiments, the power source 316 is provided separately from the wireless viewing device 300 and installed into the wireless viewing device 300 prior to the use of the wireless viewing device 300 in a procedure. In some embodiments, the power source 316 is removable from the wireless viewing device 300 for separate disposal.

In some embodiments, the power source 316 for the wireless viewing device 300 comprises a power receiver for a radio-frequency (RF)-based power system. The power receiver for an RF-based power system receives energy waveforms from a transmitter and converts the RF-based energy to DC current. In some embodiments, the power receiver comprises at least one power antenna for collecting RF-based energy waveforms from a transmitter. In further embodiments, the receiver comprises multiple power antennas for collecting RF-based energy waveforms from a power transmitter. In still further embodiments, the power receiver comprises paired power antennas for collecting RF-based energy waveforms from a power transmitter. In other still further embodiments, the power receiver comprises at least one power antenna array for collecting RF-based energy waveforms from a power transmitter. In some embodiments, the power receiver is configured to receive and convert energy waveforms from a power transmitter located at least 30 feet away from the wireless viewing device 300. In other embodiments, the power receiver is configured to receive and convert energy waveforms from a power transmitter located at least 15 feet away from the wireless viewing device 300. In still other embodiments, the power receiver is configured to receive and convert energy waveforms from a power transmitter located at least 10 feet away from the wireless viewing device 300. In yet other embodiments, the power receiver is configured to receive and convert energy waveforms from a power transmitter located at least 5 feet away from the wireless viewing device 300. In some embodiments, a power source 316 that comprises a power receiver for an RF-based power system further comprises a battery for storing energy received through and converted by the power receiver. In some embodiments, the battery is a rechargeable battery.

Still referring to FIG. 3, the wireless viewing device 300 comprises a wand 320 that is extendable through an entry portal to a target location in a subject. The wand 320 comprises a proximal end 321 and a distal end 322, wherein the proximal end 321 of the wand 320 is attached to the distal end of the housing 310. In some embodiments, the wand 320 is flexible. In other embodiments, the wand 320 is rigid. In some embodiments, the wand 320 is composed of a clear material. In further embodiments, the clear material is polycarbonate. In some embodiments, the wand 320 is marked with gradations showing how far the wand 320 had been inserted through an entry portal. In some embodiments, the wand 320 is solid. In other embodiments, the wand 320 is tubular with a hollow central lumen. In still other embodiments, the wand 320 is solid, save for channels between the proximal 321 and distal 322 ends of the wand 320 for wiring or optical fibers. In some further embodiments, the channels are internal within the wand 320. In other embodiments, the channels are indents in an external surface of the wand 320.

In some embodiments, at least one surface of the wand 320 is flattened. In other embodiments, the body of the wand 320 is generally rounded, circular, oval or elliptical. In some embodiments, the distal end 322 of the wand 320 is angled between 20 and 70 degrees. In further embodiments, the distal end 322 of the wand 320 is angled between 30 and 60 degrees. In some particular embodiments, the distal end 322 of the wand 320 is angled about 30 degrees. Further embodiments, the distal end 322 of the wand 320 is angled between 30 and 60 degrees. In other particular embodiments, the distal end 322 of the wand 320 is angled about 45 degrees. In certain embodiments, the distal end 322 comprises a leading edge that is turned upwards, allowing the wand 320 to separate and form a passage from the entry portal through/between/under/over body tissues to and/or past a target tissue. In some further embodiments, the edge can be flattened.

The wand 320 further comprises a camera 324 affixed at its distal end 322. In some embodiments, the camera 324 has a resolution of at least 100×100 pixels. In a further embodiment, the camera 324 has a resolution of at least 150×150 pixels. In a still further embodiment, the camera 324 has a resolution of at least 200×200 pixels. In an even further embodiment, the camera 324 has a resolution of at least 250×250 pixels. In some embodiments, the camera 324 is a NANEYE® camera. In some embodiments, the camera 324 is connected to the internal control board having transmitter functions 314 by wire. In some embodiments, the camera can be rotated, optically zoomed, digitally zoomed and focused either by controls on the wireless viewing device or remotely, such as by an app on a computer, smart phone or tablet. In some embodiments, the light source 312 is located at the distal end 322 of the wand 320 along with the camera 324.

In some embodiments, the wand 320 has a clear covering over the camera 324 and/or light source 312 at the distal end 322 of the wand 320. In some embodiments, the clear covering is polycarbonate. In some embodiments, the clear cover has magnifying properties.

In some embodiments, the camera 324 is located within the housing and comprises an optical fiber component that extends into the wand 320.

In some embodiments, the housing 310 fits in the palm of a hand. In some embodiments, manipulation of the entire wireless viewing device 300 can be done with a single hand.

In an alternative embodiment, the camera 324 remains in a fixed position within the housing 310. In such an embodiment, the camera 324 comprises an image transmitting optical fiber, which is attached at its proximal end to the camera 324, with the distal end of the image transmitting optical fiber being located in the proximity of the distal end 322 of the wand 320, such that from the fixed position of the image transmitting optical fiber the camera 324 can observe and image tissues through the distal end 322 of the wand 320. In another alternative embodiment, the light source 312 also remains in a fixed position within the housing 310. In such an embodiment, the light source 312 comprises a light transmitting optical fiber, which is attached at its proximal end to the light source 312, with the distal end of the light transmitting optical fiber being located in the proximity of the distal end 322 of the wand 320, such that from the fixed position of the light transmitting optical fiber the light source 312 can illuminate tissues through the distal end 322 of the wand 320.

Yet another aspect of the present application relates to a reusable wireless viewing device for observation of bodily tissues, wherein the main components are reusable and can be affixed to a disposable cannula. The main components of the wireless viewing device comprise a housing having a proximal end and a distal end; wherein the distal end is adapted to affix to the proximal end of the disposable cannula. Enclosed within the housing are a circuit board with a processor, a transmitter for wirelessly communicating with at least one external receiver; and a power source. The main components of the wireless viewing device further comprise a camera with a lens and a light source wired to the circuit board, wherein the wires protrude through an opening at the distal end of the housing such that the wires, camera and light source are completely enclosed by the disposable cannula when the cannula is affixed to the housing, with the camera and light source being located at or proximal to the distal end of the affixed cannula.

As with other embodiments of the present application, the reusable wireless viewing device of this disclosure can be used on any target tissue, bone, joint or target area of the body of a subject as set forth above. In some embodiments, the wireless viewing device of this embodiment can be used for minimally-invasive observation of an internal bodily target location. The wireless viewing device can be used, for example, for pre-treatment, or pre-operative, observation or diagnosis; post-treatment, or post-operative, observation or follow-up; or general observation of a target location in need thereof without further treatment or operative intervention. In some embodiments, the wireless viewing device of this embodiment can be used for monitoring of the progress or regression of a condition or target tissue, such as a surgical repair of a target tissue or the growth/regression of a neoplasm or tumor.

A reusable wireless viewing device as embodied by the present application has an advantage for the practitioner and the subject in that it is compact, easily transportable and the portion of the device which comes in contact with the subject, the cannula, is single use and disposable.

The device does not need to be physically attached to monitoring or viewing equipment. The monitoring or viewing equipment can be any suitable platform including, but not limited to mainframe computer, desktop computer, laptop computer, tablet or smartphone. In some embodiments, the device transmits signal to the monitoring or viewing equipment via an app. In some embodiments, the wireless communication is encrypted.

Additionally, the reusable wireless viewing device can be used independently or with a variety of devices that traverse an entry portal into the body of a subject for observation of a target tissue. Such devices that traverse an entry portal include, but are not limited to, a cannula, anoscope, port or any other suitable tubular entry device.

The reusable viewing device described herein is completely self-contained within the singular housing and disposable cannula. The present device eliminates the need for connectors or attachments such as external pods comprising additional power sources, light sources, transmitters & receivers or imaging equipment. This elimination of the need for additional components simplifies the use for the practitioner, as well as enhances the sterility of the device.

A reusable wireless viewing device of the present application is lightweight, compact and can be manipulated with a single hand. The weight of the device is less than about one pound, allowing the device to be easily carried within a pocket, backpack, satchel or case. In some embodiments, the device weighs less than about 15, 14, 13, 12, 11, 10, 9, 8, 7, 6, 5, 4 or 3 ounces.

The housing of the reusable wireless viewing device can be generally rectangular or oval in shape. The housing can also serve as a grip or handle for the device. All on-board components of the device are housed within a single housing, or within the housing and the disposable cannula. In general, the housing of the device can be easily held within the palm of one hand and manipulated by that one hand. In some embodiments, the overall dimensions of the housing (with the longest dimension being measured from the distal end to the proximal end of the housing) are less than about 7 inches in length, 2 inches in width and 2 inches in thickness (7×2×2). In further embodiments, the overall dimensions of the housing are less than about 6×2×1. In still further embodiments, the overall dimensions of the housing are less than about 5×1.5×1. In even further embodiments, the overall dimensions of the housing are less than about 5×1.5×0.5. The outer surface of the housing may be textured, grooved, indented or shaped to facilitate gripping by a hand or by another device.

The disposable cannula of the reusable wireless viewing device is attached at its proximal end to the distal end of the housing. The disposable cannula may be detachable/replaceable by any suitable means including, but not limited to, a luer-lock type system, bayonet, cam lock, snap-fit or threaded to screw into or onto the distal end of the housing. The wand can be opaque, translucent or transparent. The wand can be flexible or rigid. In some embodiments, the wand is made of polycarbonate. In some embodiments a wand of the present device is about 12 inches in length. In some embodiments, a wand of the present device is about 11, 10, 9, 8, 7, 6, 5, 4, 3 or 2 inches in length.

In some embodiments, one or more disposable cannulas may be supplied with a housing as part of a kit. Each cannula supplied with the housing may be designed or suited for a particular need for the examination and/or treatment of a subject. For example, in a kit with multiple cannulas supplied, the practitioner can select a cannula that is best suited for use with the immediate treatment/examination need of the subject. The cannulas can be interchanged during the treatment of that subject based upon changing needs by the practitioner for the treatment/examination of that subject.

In some embodiments, the housing can be enclosed in a disposable sheath, wrap or other covering.

The device can be supplied as part of a kit that includes additional instruments useful with the device such as, but not limited to, scalpel, elevator, dilator, bandages, tape, needles and sutures.

The reusable wireless viewing device can be used in a clinical setting. The clinical setting can be a hospital, emergency clinic, outpatient clinic, or office, for example. The device can also be used outside the clinical setting, such as, but not limited to, in an emergency situation. The device of the present application can be used by various practitioners including, but not limited to, a physician, surgeon, nurse, nurse practitioner, first responder, paramedic, emergency medical technician, medic, corpsman, therapist, trainer, technician or caregiver.

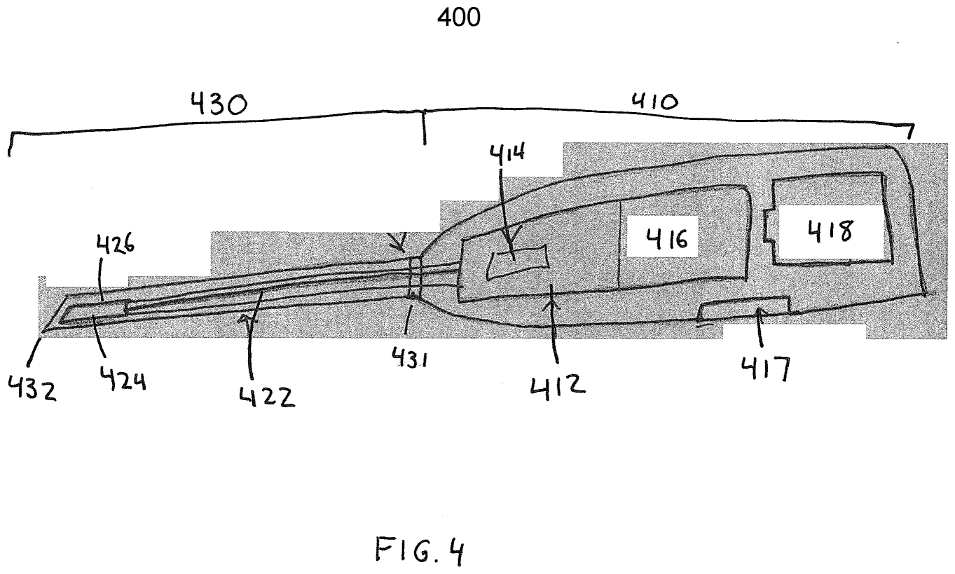

FIG. 4 illustrates an exemplary reusable wireless viewing device 400 of the present application. The wireless viewing device comprises a housing 410 that also serves as a handle for the device and a disposable cannula 430, the proximal end of which is attached to the distal end of the housing 410. In some embodiments, the device can be handheld. In some further embodiments, the housing is formed in a shape to facilitate gripping with a single hand. In some embodiments, the device can be directly controlled such as, but not limited to, manual control. In other embodiments, the device can be remotely controlled. In some embodiments, the device can be held by another device. In some embodiments, the device could be attached to, held, by, inserted into or under the control of a robotic device or tool.

Still referring to FIG. 4, the housing 410 further contains an internal circuit board 412 having a processor 414 resident thereon.

The circuit board 412 is interfaced with a wireless transmitter 416 that is capable of, for example, sending video images obtained by the wireless viewing device 400 to least one external receiver. In some embodiments, the device comprises an information display provided on the housing 410 to display frequency information of a transmission frequency of the wireless transmitter. In some embodiments, the wireless transmitter 416 also has receiver functions, such that functions of the wireless viewing device 400 can be controlled remotely. For example, functions that could be controlled remotely include, but are not limited to, camera focus, camera optical zoom, camera digital zoom, camera field of view, camera angle, camera rotation, light on/off, and light intensity.

In some embodiments, the wireless transmitter 416 is a microchip transmitter. In some embodiments, the wireless transmitter 416 and the at least one external receiver communicate via wireless local area networking based on the IEEE 802.11 standards (WiFi). In other embodiments, communication is via short-wavelength radio transmissions, for example in the ISM band from 2400-2480 MHz (IEEE 802.15.1, or Bluetooth®). In some embodiments, communication is via radio frequency communication signals (e.g., FM radio signal). In other embodiments, communication is via microwave or infrared (IR) communication signals from the wireless sensor. In other embodiments, communication is via near-field communication (NFC) signals between the internal control board having transmitter and receiver functions for wirelessly communicating and the at least one external receiver and transmitter. In certain embodiments, the wireless transmitter may utilize satellite communication. In various embodiments, the wireless transmitter utilizes wireless sensor networks such as ZigBee®, EnOcean®, TransferJet®, Ultra-wideband; or short-range point-to-point communication such as radio frequency identification (RFID). In some embodiments, communication is via digital communication. In other embodiments, communication is via analog communication.

In some embodiments, the external receiver is interfaced with a computer terminal or video monitor. In some further embodiments, the computer terminal is a notebook computer. In other embodiments, the external receiver is a tablet or smart phone. In some further embodiments, the computer, tablet or smart phone comprises an application (app) that communicates with the wireless viewing device 400. In still further embodiments, the app that communicates with the wireless viewing device 400 is a dedicated app. In some embodiments, the wireless viewing device 400 is provided with a unique identifier that can be entered into/associated with the app for dedicated communication between an individual wireless viewing device 400 and the app. In some embodiments, the app is capable of recording the transmission from the wireless viewing device as a video or individual pictures/screen captures. In further embodiments, the recordings can be saved into an archive, such as a medical record of the subject. In particular embodiments, the app is capable of remotely controlling functions of the wireless viewing device. For example, functions that could be controlled remotely include, but are not limited to, camera focus, camera optical zoom, camera digital zoom, camera field of view, camera angle, camera rotation, light on/off, and light intensity.

In other embodiments, the housing 410 further comprises a video display showing real time images from a camera 424 of the device.

In some embodiments, the wireless viewing device 400 comprises a port or slot 417 for the insertion of a memory device for storing images or information from a performed procedure, wherein said images or information can be retrieved from the device. In some embodiments, the memory device is an SD card, a micro-SD card, or a USB device, such as a flash drive.

As shown in FIG. 4, the housing further comprises a power source 418. In some embodiments, the power source 418 is a battery. In some embodiments, the battery is rechargeable. In further embodiments, the battery is a lithium battery. In some embodiments, the power source 418 is installed within the wireless viewing device 400 upon manufacture, or prior to provision to a practitioner. In other embodiments, the power source 418 is provided separately from the wireless viewing device 400 and installed into the wireless viewing device 400 prior to the use of the wireless viewing device 400 in a procedure. In some embodiments, the power source 418 is removable from the wireless viewing device 400 for separate disposal.

In some embodiments, the power source 418 for the wireless viewing device 400 comprises a power receiver for a radio-frequency (RF)-based power system. The power receiver for an RF-based power system receives energy waveforms from a transmitter and converts the RF-based energy to DC current. In some embodiments, the power receiver comprises at least one power antenna for collecting RF-based energy waveforms from a transmitter. In further embodiments, the receiver comprises multiple power antennas for collecting RF-based energy waveforms from a power transmitter. In still further embodiments, the power receiver comprises paired power antennas for collecting RF-based energy waveforms from a power transmitter. In other still further embodiments, the power receiver comprises at least one power antenna array for collecting RF-based energy waveforms from a power transmitter. In some embodiments, the power receiver is configured to receive and convert energy waveforms from a power transmitter located at least 30 feet away from the wireless viewing device 400. In other embodiments, the power receiver is configured to receive and convert energy waveforms from a power transmitter located at least 15 feet away from the wireless viewing device 400. In still other embodiments, the power receiver is configured to receive and convert energy waveforms from a power transmitter located at least 10 feet away from the wireless viewing device 400. In yet other embodiments, the power receiver is configured to receive and convert energy waveforms from a power transmitter located at least 5 feet away from the wireless viewing device 400. In some embodiments, a power source 418 that comprises a power receiver for an RF-based power system further comprises a battery for storing energy received through and converted by the power receiver.

Still referring to FIG. 4, the wireless viewing device 400 comprises wires 422, the proximal end of which attach to the circuit board 412 within the housing 410 of the device. The distal end of the wires 422 extend through an opening in the distal end of the housing 410, which is completely encircled by the proximal end 431 of the cannula 430 when the cannula 430 is attached to the housing 410 such that the entire device is sealed against fluid entry. The distal end of the wires 422 are attached to a camera 424 and a light source 426 for illumination of the camera's 424 viewing area. In some embodiments, the camera 424 has a resolution of at least 100×100 pixels. In a further embodiment, the camera 424 has a resolution of at least 150×150 pixels. In a still further embodiment, the camera 424 has a resolution of at least 200×200 pixels. In an even further embodiment, the camera 424 has a resolution of at least 250×250 pixels. In some embodiments, the camera 424 is a NANEYE® camera. In some embodiments, the camera 424 is high definition. In some embodiments, the camera 424 can be rotated, optically zoomed, digitally zoomed and focused either by controls on the wireless viewing device or remotely, such as by an app on a computer, smart phone or tablet. In some embodiments, the camera 424 is angled between 20 and 70 degrees. In some embodiments, the camera 424 is angled between 30 and 60 degrees. In some particular embodiments, the camera 424 is angled about 30 degrees. In other particular embodiments, the camera 424 is angled about 45 degrees. In some embodiments, the camera 424 is a panoramic, wide angle or 360° camera. In some embodiments, the light source 426 is an LED light source. In some embodiments, the LED light source is located within the housing and the illumination is carried to the proximity of the distal end 432 of the cannula 430.

The disposable cannula 430 comprises a proximal end 431 and a distal end 432, wherein the proximal end 431 of the wand 430 is attached to the distal end of the housing 410. In some embodiments, the cannula 430 is flexible. In other embodiments, the cannula 430 is rigid. In some embodiments, the cannula 430 is composed of a clear material. In further embodiments, the clear material is polycarbonate. In some embodiments, the cannula 430 is marked with gradations showing how far the cannula 430 had been inserted through an entry portal.

In some embodiments, at least one surface of the cannula 430 is flattened. In other embodiments, the body of the cannula 430 is generally rounded, circular, oval or elliptical. In some embodiments, the distal end 432 of the cannula 430 is angled between 20 and 70 degrees. In further embodiments, the distal end 432 of the cannula 430 is angled between 30 and 60 degrees. In some particular embodiments, the distal end 432 of the cannula 430 is angled about 30 degrees. In other particular embodiments, the distal end 432 of the cannula 430 is angled about 45 degrees. In certain embodiments, the distal end 432 comprises a leading edge that is turned upwards, allowing the cannula 430 to separate and form a passage from the entry portal through/between/under/over body tissues to and/or past a target tissue. In some further embodiments, the edge can be flattened.

In some embodiments, the cannula 430 has a clear covering over the camera 424 and/or light source 426 at the distal end 432 of the cannula 430. In some embodiments, the clear covering is polycarbonate. In some embodiments, the clear cover has magnifying properties.

The portion of the wires 422 protruding from the distal end of the case, camera 424 and light source 426 are completely enclosed within the cannula 430 when the proximal end 431 of the cannula 430 is attached to the distal end of the housing 410. In this manner, the wires 422, camera 424 and light source 426 are physically isolated from direct contact with the subject during a procedure.

In alternative embodiments, the camera 424 is located within the housing and comprises an optical fiber and lens component that extends into the cannula 430.

In some embodiments, the housing 410 fits in the palm of a hand. In some embodiments, manipulation of the entire wireless viewing device 400 can be done with a single hand.

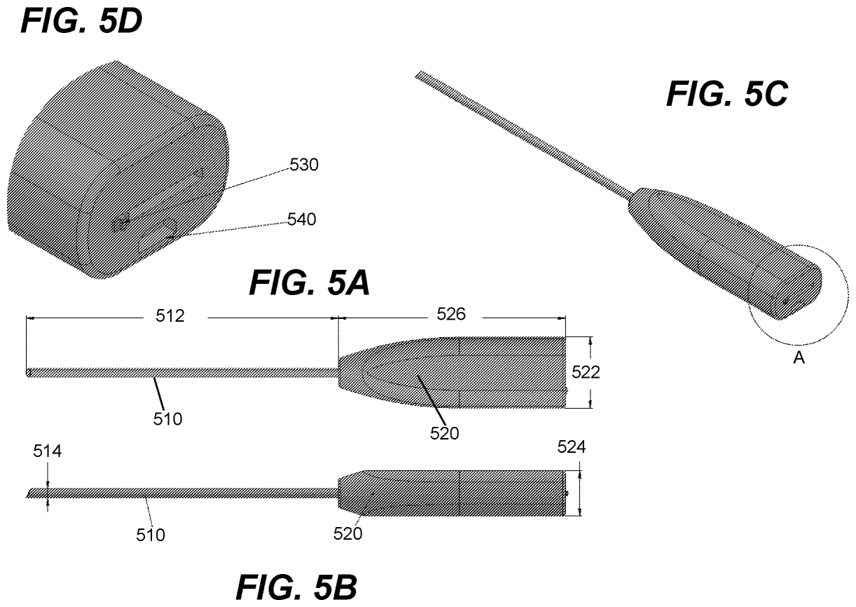

FIG. 5A depicts a top view an embodiment of a viewing device of the present application wherein the cannula 510 and housing/hand grip 520 are joined as a single unit. In some embodiments, the housing 520 has a width 522 of between about 20 mm and 40 mm. In other embodiments, the housing 520 has a width 522 of between about 25 mm and 35 mm. In particular embodiments, the housing 520 has a width 522 of about 30 mm. In some embodiments, the housing 520 has a length 526 of about 80 mm to about 150 mm. In some embodiments, the housing 520 has a length 526 of about 80 mm to about 120 mm. In some embodiments, the housing 520 has a length 526 of about 90 mm to about 110 mm. In particular embodiments, the housing 520 has a length 526 of about 100 mm.

Also in FIG. 5A, in some embodiments, the cannula 510 has a length 512 of between about 50 mm and about 250 mm. In some embodiments, the cannula 510 has a length 512 of between about 80 mm and about 200 mm. In some embodiments, the cannula 510 has a length 512 of between about 100 mm and about 180 mm. In particular embodiments, the cannula 510 has a length 512 of about 140 mm. In other embodiments, dependent upon the intended use or target tissue, the cannula 510 has a length 512 of about 50, 60, 70, 80, 90, 100, 110, 120, 130, 150, 160, 170, 180, 190, 200, 210, 220, 230, 240, or 250 mm.

In some embodiments, the cannula 510 is made of a polymer material. In some further embodiments, the polymer material is clear, such as polycarbonate. In other embodiments, the cannula 510 is made of an alloy material. In some embodiments, the cannula 510 is made of stainless steel.

FIG. 5B depicts a side view of the embodiment of FIG. 5A. In some embodiments, the housing 520 has a height 524 of between about 10 mm and 30 mm. In other embodiments, the housing 520 has a height 524 of between about 15 mm and 25 mm. In particular embodiments, the housing 520 has a height 524 of about 20 mm.

In some embodiments, the cannula 510 has a diameter 514 of between about 2 mm and about 10 mm. In some embodiments, the cannula 510 has a diameter 514 of between about 2 mm and about 6 mm. In some embodiments, the cannula 510 has a diameter 514 of between about 3 mm and about 5 mm. In particular embodiments, the cannula 510 has a diameter 514 of about 4 mm.

FIG. 5C shows a perspective view of the embodiment in FIG. 5A. FIG. 5D is an expanded view of the area marked “A” in FIG. 5C. In some embodiments, the housing 520 comprises a switch 530 for controlling the light source, such as an LED, of the device. In some embodiments, the switch 530 is on a proximal surface of the housing 520. In other embodiments, the switch 530 is on a top, bottom or side surface of the housing 520. In some embodiments, the switch 530 controls on/off function of the light source. In some embodiments, the switch 530 controls the brightness or intensity of the light source. In particular embodiments, the switch 530 controls both on/off function and the brightness or intensity of the light source. In some embodiments, the switch 530 is a slider, as depicted. In other embodiments, the switch 530 is a rocker switch or a thumbwheel.

Also, still in FIG. 5D, in some embodiments, the housing 520 further comprises a port 540 for the attachment of a data or controller cable. In some embodiments, the port 540 is on a proximal surface of the housing 520. In other embodiments, the port 540 is on a top, bottom or side surface of the housing 520. In some embodiments, the port 540 accommodates a micro-USB connector. In other embodiments, the port 540 accommodates a mini-USB, USB-C or lightning connector. In still other embodiments, the housing 520 comprises a slot for the insertion of a memory device selected from the group consisting of an SD card, a micro-SD card, a USB device and a flash drive. In some embodiments, the slot is in addition to the port 540. In other embodiments, the slot takes the place of the port 540.

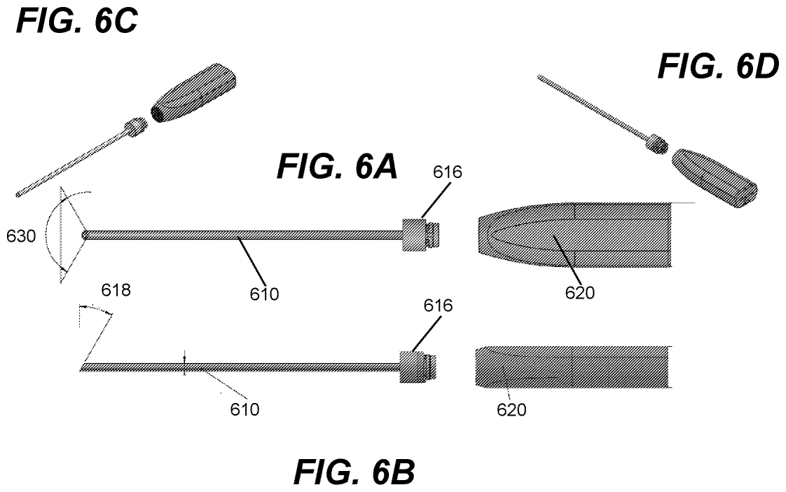

Turning now to FIG. 6A, depicted is an embodiment of the viewing device having a detachable cannula 610. In most embodiments, dimensions of the device depicted in FIGS. 6A-6D are similar to the dimensions described in FIGS. 5A-5D.

The detachable cannula 610 can be replaced with different detachable cannulas 610 of differing lengths, diameters or shapes dependent upon the application while still using a common housing/hand grip 620 component. Additionally, a detachable cannula 610 allows for having a disposable cannula 610 in use with a durable housing 620 that can be sterilized between uses.

The detachable cannula 610 comprises a coupling 616 for attaching the catheter 610 to the distal end of the housing 620. The coupling 616 can be of any suitable type for securing the cannula 610 to the housing 620 including, but not limited to, bayonet, snap, tongue and groove, screw-in, luer lock, and slip.

In some embodiments, the distal end of the cannula 610 provides a field of view 630 for the optical sensor or camera of between about 90 degrees and 180 degrees. In some embodiments, the field of view 630 is between about 90 degrees and 150 degrees. In some embodiments, the field of view 630 is between about 100 degrees and 140 degrees. In some embodiments, the field of view 630 is between about 110 degrees and 130 degrees. In particular embodiments the field of view is about 120 degrees. In other embodiments the field of view is about 90, 100, 110, 130, 140, 150, 160, 170, or 180 degrees.

FIG. 6B shows that, in some embodiments, the distal end is angled 618 back between about 15 degrees and about 45 degrees. In some embodiments, the angle 618 is between about 20 degrees and about 40 degrees. In some embodiments, the angle 618 is between about 25 degrees and about 35 degrees. In some embodiments, the angle 618 is about 30 degrees.

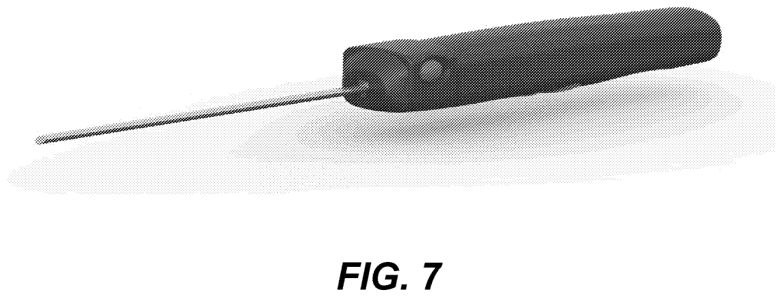

FIG. 7 shows another embodiment 700 of the wireless viewing device of the present application.

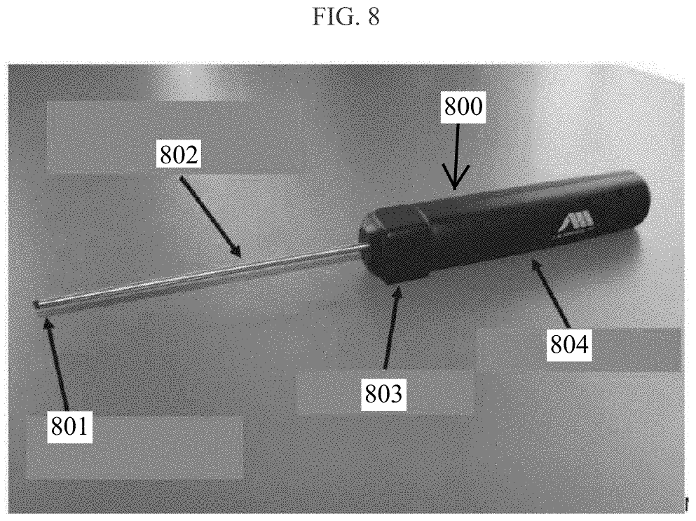

FIG. 8 shows another embodiment of the wireless viewing device 800 of the present application. In one embodiment, the wireless viewing device 800 comprises a camera module 801, a shaft 802, an on/off button 803, and a scope handpiece 804. The camera module 801 may include a camera and a light source, or the camera module 801 may include a camera only, with a light source being externally added. The light source can be an LED light source or any other light source deemed suitable. The on/off button 803 may be switched to “on” position to allow the camera in the camera module 801 to turn on separately from the light source, or it may be switched to “on” position to allow the camera and the light source to activate simultaneously. In some embodiments, the scope handpiece 804 comprises a microcontroller used for controlling the movement and positioning of the camera module 801 and other surgical tools such as a probe, a knife, a cautery, etc.

In other embodiments, the device may comprise a rotatable camera included in the camera module 801. The rotatable camera may be enclosed within clear transparent material which allows the camera to film moving or still images of surrounding tissue without the need to either bend the tip of the device or rotate the angle of the device itself. In specific embodiments, the camera may be able to take images that are forward-facing, rear-facing or both. In specific embodiments, the camera may be equipped with one or more lens allowing greater field of view for filming. In particular embodiments, the camera can transmit multiple images from different perspectives at the same time, which may be displayed, for example, as split screen images on a computer screen, or other televisual display screen.

The shaft 802 can be made of stainless steel, a plastic material, or any other material considered appropriate. The material used for the shaft 802 can be opaque, transparent, or partially opaque/transparent. The material used for the shaft 802 may be able to endure elastic deformation. The shaft 802 may be able to reversibly twist its tubular body up to 90 degrees in the circumferential direction of the shaft 802. Moreover, the shaft 802 may be capable of reversible bending of the distal end of the shaft 802 up to 45 degrees respective to the axis of the shaft 802. Further, the shaft 802 may be capable of reversible compression or expansion of its tubular body up to 50% of its diameter in any radial direction.

In some embodiments, the shaft 802 has a clear covering over the camera module 801 at the distal end of the shaft 802. In some embodiments, the clear covering is polycarbonate. In some embodiments, the clear covering has magnifying properties. For example, the clear covering may be a magnifying glass or plastic that has magnifying properties. In other embodiments, the camera in the camera module 801 has a capacity to magnify the recorder image. The resolution of the camera in the camera module 801 can be adjustable. The recorded image may be magnified by increasing the camera resolution and/or by zooming into an area of interest. Functions of the camera module 801 that could be controlled electronically and/or remotely include, but are not limited to, camera focus, camera optical zoom, camera digital zoom, camera field of view, camera angle, camera rotation, light on/off, and light intensity.

In certain embodiments, selected tools, such as a probe, a camera, a blade, a cautery or other suitable surgical tools are moved into and out of the shaft 802. In some embodiments, a housing of the scope handpiece 804 further comprises a rocker switch that is connected to the microcontroller and used to move the selected tools, such as the probe, the camera, the blade, the cautery or other suitable surgical tools within the housing into and out of the shaft 802. In other embodiments, the movement of tools such as the probe, the camera, the blade, the cautery or other suitable surgical tools within the housing into and out of the shaft 802 is controlled electronically and/or remotely.

In certain embodiments, the camera module 801 comprises a video flexible scope. The device is intended to be used in an operating room during endoscopic procedures. The device is suitable for use in diagnostic and operative endoscopic procedures to provide illumination and visualization of an interior cavity of the body through either a natural or surgical opening. In certain embodiments, the camera module may also have a transmitter which may transmit signal to a wireless receiver. In some embodiment, the wireless receiver is located in a viewing device. In some embodiments, the wireless receiver is located in the handpiece of the device 800, which further transmits the signals received by the wireless receiver to a secondary receiver located outside the device 800. In other embodiments, the camera module is connected to a transmitter housed in the handpiece 804 by a wire and/or optical fiber. In particular embodiments, the camera module comprises a video flexible scope, LED light source and microprocessor.

In one example, the wireless viewing device 800 further comprises an integral power source to provide energy for the camera module 801, the light source contained in the camera module 801, a circuit board and any mechanical functions within the wireless viewing device 800. In some embodiments, the power source is a battery. In further embodiments, the battery is a lithium battery. In one instance, the power source is contained in the handpiece 804 and a wire connection is used to connect the power source to the camera module. In another instance, the power source is contained in the camera module 801.

In some embodiments, the power source is installed within the device 800 upon manufacture, or prior to provision to a practitioner. In other embodiments, the power source is provided separately from the device 800 and installed into the device 800 prior to the use of the device 800 in a surgical procedure. In some embodiments, the power source is removable from the device 800 for separate disposal.

In one example, an electric motor is contained in the handpiece 804 of the wireless viewing device 800 and powered by the power source. In one example, mechanical structures that connect the camera module 801 to the electric motor in the handpiece 804 are accommodated in the shaft 802 to transfer axial and rotary forces from the electric motor to the camera module 801. The wire connection and the mechanical structures between the camera module 801 and the handpiece 804 are used for controlling the camera focus, camera optical zoom, camera digital zoom, camera field of view, camera angle, camera rotation, light on/off, and light intensity.

In certain instances, the wireless viewing device 800 is controlled from a remote control unit and instructions are wirelessly sent to the electric motor and/or the microcontroller. The wireless instructions sent to the electric motor determine camera field of view, camera angle, camera rotation, or any other positioning parameters of the camera module 801. The wireless instructions sent to the microcontroller set the camera focus, camera optical zoom, camera digital zoom, light on/off, and light intensity, among other data acquisition parameters. In some embodiments, the instructions wirelessly sent to the wireless viewing device 800 control movement of the probe, the camera, the blade, the cautery or other suitable surgical tools within the housing of the handpiece 804 into and out of the shaft 802.

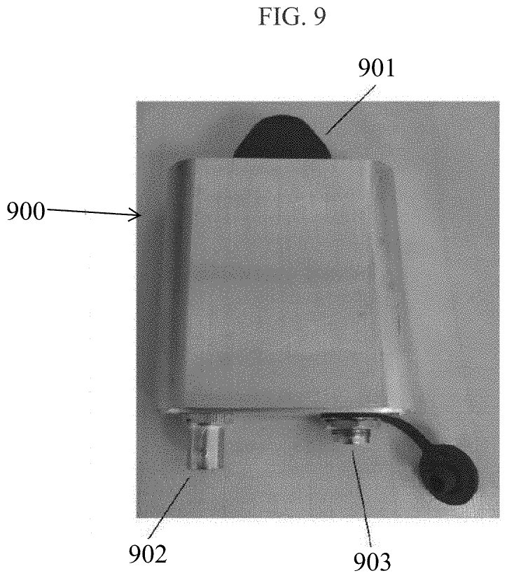

FIG. 9 shows an embodiment 900 of the receiver with an antenna 901, a video output 902 and a power input 903. In a specific embodiment, the receiver has an NC video output and a TV power input. The receiver is then connected to a monitor or other suitable electronic display instrumentality. In certain embodiments, the receiver is a diversity receiver (a receiver with multiple antennas with the ability to switch, so as to enhance the signal quality). In other embodiments, the receiver is a receiver plus monitor combination. In further embodiments the receiver is a receiver plus digital video recorder combination. In other embodiments, the receiver is a receiver with video output. In particular embodiments, the receiver may be a receiver plus monitor on a wearable watch.

In certain embodiments, the wireless endoscopy system herein is a sterile, disposable, endoscopic device that consists of a wireless camera module 801 and receiver 900. The endoscopy unit incorporates a transmitter that delivers an uncompressed analog video signal to the receiver 900. In certain embodiments, the camera module 801 comprises a transmitter that transmits visual information to the receiver 900 in the handpiece 804, from where the data is transmitted wirelessly. In certain embodiments, the camera module 801 comprises a micro-circuit board and the acquired data is transmitted directly from the camera module 801 to the remotely located receiver 900.

In certain embodiments, the receiver module 900 is connected to a video display using a standard TV composite cable. In some embodiments, the wireless viewing device 800 does not include a monitor mounted on the housing of the handpiece 804. In some instances, a monitor is located apart from the wireless viewing device 800 for remote monitoring of the acquired visual information.

In certain embodiments, an integrated LED light source eliminates the need for a separate light source and light cable, and the camera is powered by battery. In particular embodiments, the device comprises the endoscopy unit with the LED light source and camera embedded in the shaft, preferably embedded at the distal end of the shaft, a wireless receiver, a composite video cable, and power cords. In certain embodiments, the handheld camera unit is disposable equipment.

In certain embodiments, the receiver 900 is placed in an area such that it is no more than fifteen feet away from the endoscopy unit for the duration of the procedure. Wires are cleared of any walking path to minimize risk of tripping. The composite cable is connected from the output of the receiver 900 to the composite input on the video monitor. The barrel connector of the AC power supply is connected to the TV input on the receiver 900. Once the receiver has been successfully set up, the endoscopy unit may be used to provide visualization for minimally invasive endoscopic surgical procedures.

In another embodiment, the device is an endoscope that can wirelessly network with a remote receiver. The remote receiver may be any suitable electronic device for receiving wirelessly transmitted information, including, but not limited to, devices such as: mobile phones, electronic notepads, electronic tablets, electronic automobile dashboards (e.g., in ambulances or cars used for medical-related purposes), electronic motorcycle dashboards, electronic wristbands, electronic neckwear, wall-mounted screens, portable monitors (e.g. wheeled monitors in medical facilities), electronic headbands, electronic helmets, electronic eyewear (e.g. glasses with lens that can display information in real time to the wearer), electronic rings, networked computers (e.g. personal computers), cloud-based computer networks, mainframes, remote viewing technology (e.g. rural doctor client-patient communication devices) and portable electronic devices in general. There may be one or more remote receivers used in some circumstances, such as for multiple doctors or other medical professionals to consult on the status of a patient.

Remote receivers may be located at the same location as the patient, e.g., the same room or a nearby room, or may be located some distance away. In certain instances the remote receiver may both receive and then boost the wireless signal so that one or more secondary receivers located at other locations from the patient, e.g., a different city or country, can also receive the wireless information being transmitted by the device.

In certain embodiments, wireless information, e.g. images, transmitted by the device may be encrypted so as to ensure patient confidentiality. In particular embodiments, the wireless information transmitted by the device may be password-protected and the password must be used before access to the wireless information, e.g. images, can be accessed.

In certain embodiments, the wireless information, e.g. images, transmitted by the device may include additional data, such as time stamp (date, time) or identification information for the patient (e.g. hospital admission number or similar patient identification code, or name, etc.).

In a particular embodiment, for both flexible and rigid endoscopic systems using the device, the device is connected to a system that includes stereoscopic high-definition video presented via a wearable head-up display to all members of an operating team.

In further embodiments, artificial intelligence may be used to electronically control and direct the use of the device, e.g., navigation, lens cleaning, instrument manipulation, information transmitted to a receiver. In other embodiments, artificial intelligence techniques like machine learning, or deep learning, that can be networked with the device allow for expedited processing of large-volume unstructured data, and in doing so enables machines to assist clinicians in important tasks, e.g., polyp detection and classification.

In certain embodiments, to begin, the On/Off button 803 is pressed once on the unit. This button 803 is found on the side of the scope handpiece 804. When the device 800 is turned on, the LED will illuminate. The video monitor is checked to confirm that the receiver is obtaining a clear, uninterrupted signal from the camera. White balance and brightness are automatically adjusted. If the signal is blurry, there may be dust or other particulate matter blocking the camera lens, which can be cleared by wiping it with a sterile lint-free cloth. If the signal is cutting out or the picture is grainy, the receiver should be moved within less than 15 feet from the camera. In certain instances, it may be necessary to remove objects from the path between the endoscopy unit and receiver.

In some embodiments, a blade is introduced into the shaft 802 for surgical operations. In some embodiments, the blade is introduced by an endoscope as a blade mounted at the tip of an endoscope. In some embodiments, the blade is introduced as a knife assembly that is pushed into the shaft 802 by a wire, rod or tube. In some embodiment, the handpiece 804 further comprises a locking mechanism that, when employed in a locking position, immobilizes the wire, rod or tube to prevent any movement of the knife assembly or the blade relative to the shaft 802. In some embodiments, a blade is attached at the tip of the shaft 802. In other embodiments, a blade is permanently affixed to the shaft 802 for tissue engagement purposes.

For surgeries expected to last longer than 2 hours, when the unit is not in use during surgery the On/Off button 803 is pressed once to shut down the endoscopy unit in order to extend the battery life. The On/Off button 803 is pressed again to reactivate the device and ensure the video monitor displays a clear signal from the camera. Once the procedure has concluded, the On/Off button 803 is pressed once more to shut down the endoscopy unit and dispose of the device. The LED light is confirmed off to ensure the device is completely off prior to disposal. The device is not re-used and is not re-sterilizable.

In certain embodiments, the device 800 is delivered sterile. The device 800 is designed for one-time use only and cannot be re-sterilized. The receiver 900 is reusable after disinfection between procedures. To clean the receiver 900, wipe it down with a disinfecting wipe. The device and components may be stored at normal room temperature.

One of ordinary skill will understand certain features determine the optical characteristics of an endoscope. Such features include the diameter, angle of inclination, and field of view.

The angle of inclination, which is the angle between the axis of the endoscope and a line perpendicular to the surface of the lens, varies from 0 to 120 degrees. In different embodiments, the angle of inclination of the endoscopic lens of the device may be one selected from the group of less than 10 degrees, less than 20 degrees, less than 30 degrees, less than 40 degrees, less than 50 degrees, less than 60 degrees, less than 70 degrees, less than 80 degrees, less than 90 degrees. In alternative embodiments, the angle of inclination of the endoscopic lens of the device may be one selected from the group of greater than 10 degrees, greater than 20 degrees, greater than 30 degrees, greater than 40 degrees, greater than 50 degrees, greater than 60 degrees, greater than 70 degrees, greater than 80 degrees, greater than 90 degrees; but under 120 degrees. In certain embodiments, the angle of inclination of the endoscopic lens of the device may be one selected from the group of less than 100 degrees, less than 110 degrees, less than 120 degrees, or greater than 100 degrees, greater than 110 degrees; but under 120 degrees. For example, 25- and 30-degree endoscopes are most commonly used; or 70- and 90-degree endoscopes are useful in seeing around corners, such as the posterior compartments of the knee through the intercondylar notch. In certain preferred embodiments, the device is a 30 degree scope. In other embodiments, the device is a 70 degree scope. In further embodiments, the device is a 0 degree scope.

In certain embodiments that endoscopic diameter of the device ranges from 1.5 mm to 7.5 mm. In specific embodiments, the endoscopic diameter of the device may be one selected from the group of greater than 1.5 mm, greater than 2 mm, greater than 2.5 mm, greater than 3 mm, greater than 3.5 mm, greater than 4 mm, greater than 4.5 mm, greater than 5 mm, greater than 5.5 mm, greater than 6 mm, greater than 6.5 mm, greater than 7 mm, greater than 7.5 mm; but under 8 mm. In alternative embodiment, the endoscopic diameter of the device may be one selected from the group of lesser than 1.5 mm, lesser than 2 mm, lesser than 2.5 mm, lesser than 3 mm, lesser than 3.5 mm, lesser than 4 mm, lesser than 4.5 mm, lesser than 5 mm, lesser than 5.5 mm, lesser than 6 mm, lesser than 6.5 mm, lesser than 7 mm, lesser than 7.5 mm.

Field of view refers to the viewing angle encompassed by the lens and varies according to the type of endoscope, e.g., a 1.9-mm scope may have a 65-degree field of view; a 2.7-mm scope, a 90-degree field of view; a 4.0-mm scope, a 115-degree field of view. Wider viewing angles enable easier orientation by the observer. Rotation of the forward oblique viewing (25- and 30-degree) endoscopes allows a much larger area of a targeted area, e.g. a joint, to be observed. One of ordinary skill will understand that a desired field of view may be incorporated into the design of the device.

In certain embodiments, the camera is a video flexible scope (chip-on-tip camera). A distal chip-on-tip camera is a small, often sub-millimeter, camera that can be integrated into the device 800. For example, Toshiba® Imaging's IK-CT2 is an ultra-small chip-on-tip video camera system with a 0.7×0.7 mm back-side illuminated CMOS sensor featuring 220×220 pixel resolution, and may also include an LED lighting option. Other examples are chip-on-tip cameras produced by Omnivision®, e.g., CameraCube, CameraChip, OmniBSI+, OmniBSI-2, OmniPixel2, OmniPixel3-GS, OmniPixel3-HS, OmniPixel3, PureCel, TrueFocus™. Further examples are the Naneye® camera by Omosis, or the Microscout® camera produced by Medigus.

In certain embodiments, cameras using three-chip technology allow even greater color resolution, and digitalization of the video signal results in high-quality imaging. In one embodiment, a camera has an imaging system that uses three separate charge-coupled devices (CCDs), each one receiving filtered red, green, or blue color ranges. Light coming in from the lens is split by a complex prism into three beams, which are then filtered to produce colored light in three color ranges or “bands”. In certain embodiments, the CCD is a device for the movement of electrical charge, usually from within the device to an area where the charge can be manipulated, for example conversion into a digital value. This is achieved by “shifting” the signals between stages within the device one at a time. CCDs move charge between capacitive bins in the device, with the shift allowing for the transfer of charge between bins. The CCD functions as an image sensor in which pixels are represented by p-doped metal-oxide-semiconductors (MOS) capacitors. These capacitors are biased above the threshold for inversion when image acquisition begins, allowing the conversion of incoming photons into electron charges at the semiconductor-oxide interface; the CCD is then used to read out these charges.

One of ordinary skill will understand that the device may be used with CCD sensors or CMOS sensors (complementary meta-oxide sensors). In certain embodiments, a CMOS sensor (also known as an active-pixel sensor) is an image sensor where each pixel (picture element) has a photodetector and an active amplifier.

In certain embodiments, the device has a movable video flexible scope (chip-on-tip camera) combined with motorized spindles within the device that are able to change the angle of view without rotating the device because camera is located inside a flexible tip capable of bending in all directions. In particular embodiments, the device has an optical system that includes a digital image sensor supported by miniature linear motors that can move optical lens for focusing and zooming the image that is transmitted by the device. In specific embodiments, an actuator is integrated between the optics and the image processing chip. One or more actuators may be piezomotors or magnetic miniature drives.

Color co-site sampling may be used which is a system of photographic color sensing, wherein 4, 16 or 36 images are collected from the sensor and merged to form a single image. Each subsequent image physically moves the sensor by exactly one pixel, in order to collect R, G and B data for each pixel; this is known as microscanning. In further embodiments, the device may include microscanning technology, e.g., for fluorescent microscopy.

In certain embodiments, the device may be wirelessly connected to 3D goggles that may be worn by a medical professional or other individual providing treatment. In such embodiments, the wirelessly transmitted information, e.g., images, from the device are translated into a 3D image within the goggles.

In certain embodiments, the size of the sensor further reduces the diameter range of the device, e.g., chip-on-tip sensors can reduce the diameter range of the device to 1 mm diameter. This enhances the ability to perform minimally invasive surgery.

Examples of pixel size used in chip-on-tip sensor technology that may be used in the device includes, but is not limited to, 1.1 μm×1.1 μm, 1.12 μm×1.12 μm, 1.34 μm×1.34 μm, 1.4 μm×1.4 μm, 1.75 μm×1.75 μm, 2 μm×2 μm, 2.2 μm×2.2 μm, 2.5 μm×2.5 μm, 2.8 μm×2.8 μm, 3 μm×3 μm, 3.75 μm×3.75 μm, 4.2 μm×4.2 μm, 4.5 μm×4.5 μm, 6 μm×6 μm.

Examples of active pixel array characteristics used in chip-on-tip sensor technology that may be used in the device includes, but is not limited to, 328H×250V, 400H×400V, 640H×480V, 648H×488V, 656H×488V, 656H×492V, 656H×496V, 672H×492V, 752H×480V, 752H×548V, 768H×506V, 768H×576V, 1280H×1080V, 1280H×720V, 1280H×800V, 1280H×800V, 1280H×960V, 1296H×808V, 1296H×812V, 1304H×1036V, 1312H×732V, 1600H×1216V, 1624H×1212V, 1632H×1212V, 1920H×1080V, 1932H×1092V, 1952H×1096V, 2048H×1536V, 2592H×1922V, 2592H×1944V, 2688H×1520V, 2688H×1944V, 3264H×2448V, 3280H×2464V, 3296H×2460V, 3488H×2616V, 3840H×2160V, 4224H×3136V, 4256H×3016V, 4256H×3168V, 4320H×2430V, 4320H×2432V, 4416H×3312V.

Examples of frames per second used in chip-on-tip sensor technology that may be used in the device includes, but is not limited to, 15 frames per second, 30 frames per second, 120 frames per second, or greater.

In a specific embodiments the depth of field is 5 mm-50 mm, field of view is 120°, direction of view is 30°, wireless signal type is analog RF, frequency band is 5.8 GHz, image latency is <100 ms, and camera native resolution is 400×400. The system complies with IEC 60601-1, IEC 60601-2-18, IEC 60601-1, IEC 60601-2-18, IEC 60601-1-2:2007.

### Kit

Another aspect of the present application relates to an instrument kit for wireless observation of a target tissue within the body of a subject in need thereof. The kit comprises the reusable wireless viewing device of the present application. In some embodiments, the instrument kit comprises the receiver of the present application.

In some embodiments, the instrument kit comprises additional components and implements useful for wireless observation of a target tissue within the body of a subject in need thereof.

In some embodiments, the instrument kit comprises a cannula. In further embodiments, the distal end of the cannula is angled upwards. In other embodiments, the distal end of the cannula comprises an edge for separating tissues.

In other further embodiments, the cannula is composed of a clear material, such as polycarbonate. In still further embodiments, the cannula comprises at least one wing, flange or handle at or near the proximal end.

In another embodiment, the instrument kit further includes a scalpel. In yet another embodiment, the instrument kit further includes an obturator used as a path opener.

In another embodiment, the instrument kit further includes at least one retractor for holding open an entry portal.

In another embodiment, the instrument kit further includes suture material and or at least one bandage.

In another embodiment, the instrument kit comprises a power source for the wireless viewing device of the present application. In a further embodiment, the power source is a battery.

### Method for Endoscopic Observation

Another aspect of the present application relates to a method for uniportal endoscopic observation of a target tissue using the reusable wireless viewing device of the present application. Uniportal endoscopic observation allows the practitioner to visualize a target tissue and its surrounding tissues as well. In some embodiments, a practitioner may perform a surgical procedure through the same entry portal before or after the observation. In some instances, the entry portal may be a natural opening, while in other instances the entry portal is an incision. In the case of an incision, generally only a single small incision must be made. In particular embodiments, the incision is less than or equal to about 2 cm in length. In more particular embodiments, the incision is less than or equal to about 1.5 cm in length. In still more particular embodiments, the incision is less than or equal to about 1 cm in length. The single small incision allows the patient to recover more quickly and begin therapy and/or resume normal activity as tolerated sooner. In certain embodiments, a camera module observes images of the surgical area which are input from the camera module to a transmitter housed inside the device, either at the distal end in conjunction with the camera module or within the housing of the handle of the device. In a particular embodiment, images from the transmitter are transmitted to a wireless receiver that is connected to an electronic instrumentality for displaying the images.

Uniportal endoscopic surgical procedures which can be performed using the same entry portal as the reusable wireless viewing device described herein can include a number of different surgical procedures including, but not limited to, carpal tunnel release, Guyon's canal (or canal) release, cubital tunnel release, plantar fascia release, lateral release for patella realignment, release of radial tunnel, release of pronator teres, release of trigger finger, release of lacertus fibrosus, release of the extensor tendons for lateral epicondylitis, release of medial epicondylitis, release of the posterior and other compartments of the leg, forearm fascia release for fascial compartment syndrome, release of fascial compartments in the upper or lower extremities, relieving the compression of a nerve by a ligament pulley or tunnel, releasing the travel of a ligament through a ligament pulley or tunnel, surgical procedures on the spine, such as endoscopic discectomy for the treatment of degenerative disc disease, herniated discs, bulging discs, pinched nerves or sciatica, endoscopic procedures on cranial and facial tissues, fasciotomy release and blood vessel harvesting.

One embodiment of the present application relates to a method for a performing a uniportal endoscopic observation of a target tissue in a subject. Generally, the procedure requires the establishment of an entry portal. In some embodiments of the present application, the entry portal is established to the proximate side of the target tissue. In other embodiments of the present application, the entry portal is established to the distal side of the target tissue.

In some embodiments, the establishing an entry portal comprises making an incision.

In some embodiments, following the establishment of an entry portal, the distal end of the cannula portion of the device is inserted through the portal to establish an opening in the underlying tissue between the portal and the target tissue. In some embodiments, the distal end of the cannula portion of the device comprises a front edge for separating tissues.

The camera is used to view the target tissue and the surrounding tissues.

In some embodiments, the method comprises the steps of establishing an entry portal having access to the target site; inserting the distal end of a disposable cannula of a reusable wireless viewing device through the entry portal, wherein the device comprises a durable housing having a proximal end and a distal end; a circuit board having a processor, a transmitter for wirelessly communicating with at least one external receiver, and a power source, wherein the circuit board, transmitter, and power source are enclosed within the housing; wherein wires connected at their proximal ends to the circuit boards extend out through a distal opening in the housing, connecting at their distal ends to a camera or a light source. The device further comprises a disposable cannula having an open proximal end and a closed distal end, wherein the proximal end of the cannula releasably attaches to the distal end of the housing; wherein the open proximal end of the cannula releasably attaches to the housing such that the camera, light source and portion of the wires outside the housing are encased within the cannula; advancing the cannula toward the target site; and imaging the target site with the camera.

The present invention is further illustrated by the following examples which should not be construed as limiting. The contents of all references, patents and published patent applications cited throughout this application, as well as the Figures, are incorporated herein by reference.

### Example 1. Endoscopic Visualization of a Target Tissue

In a patient presenting with potential tear of a tendon requiring visual inspection, an incision is made proximate to the proximal or distal edge of the target tendon to establish an entry portal.

A dissector is introduced into the entry portal to form a pathway extending across the target tendon to at least the margin of the tendon distal to the entry portal. Once the pathway is created and the dissector removed, the distal end of a disposable cannula of a reusable wireless viewing device is introduced into the same pathway.

The wireless viewing device is activated and the camera is used to visually inspect the condition of the tendon and then withdrawn from the pathway. The cannula is removed from the housing and discarded as medical waste.

The wound is closed and a soft bandage is applied.

### Example 2. Endoscopic Visualization of a Target Tissue with Insertion into a Separate Cannula

In a patient presenting with potential tear of a tendon requiring visual inspection, an incision is made proximate to the proximal or distal edge of the target tendon to establish an entry portal.

A clear cannula is prepared for insertion through the entry portal by introducing an obturator into the lumen of the cannula. A dissector is introduced into the entry portal to form a pathway extending across the target tendon to at least the margin of the tendon distal to the entry portal. Once the pathway is created and the dissector removed, the obturator and the cannula are introduced into the same pathway. Following insertion, the obturator is withdrawn from the lumen of the cannula.

The wireless viewing device is activated and the distal end of the wand is inserted into the cannula. The wand is then advanced distally through the cannula. The camera is used to visually inspect the condition of the tendon and then withdrawn from the cannula. The cannula is then withdrawn from the pathway and entry portal.

### Example 3. Activating Wireless Endoscope

The operating room staff connect the receiver to a monitor and turn on a wireless endoscope. The scope will link to the receiver and begin to transmit analog video data over 5.8 GHz radio frequency

The above description is for the purpose of teaching the person of ordinary skill in the art how to practice the present invention, and it is not intended to detail all those obvious modifications and variations of it which will become apparent to the skilled worker upon reading the description. It is intended, however, that all such obvious modifications and variations be included within the scope of the present invention, which is defined by the following claims. The aspects and embodiments are intended to cover the components and steps in any sequence which is effective to meet the objectives there intended, unless the context specifically indicates the contrary.

What is claimed is:

1. A wireless endoscopic viewing device, comprising:
a housing having a proximal end and a distal end; a circuit board having a processor, a transmitter for wirelessly communicating with at least one external receiver, and a power source, wherein the circuit board, transmitter, and power source are enclosed within the housing;
a shaft having a proximal end and a distal end, wherein the proximal end of the shaft is attached to the distal end of the housing; and
a camera attached to the distal end of the shaft,
wherein the camera comprises a video flexible scope.

2. The device of claim 1, wherein the camera is connected to the circuit board through a wire.

3. The device of claim 1, wherein the camera comprises a microchip.

4. The device of claim 1, wherein the transmitter is a microchip transmitter.

5. The device of claim 1, wherein the camera comprises a lens with a diameter of less than 5 mm.

6. The device of claim 5, wherein the camera comprise a lens with a diameter of less than 1 mm.

7. The device of claim 1, wherein the camera comprises an LED light source.

8. The device of claim 1, wherein the shaft is made from a rigid material.

9. The device of claim 1, wherein the shaft is made from a flexible material.

10. The device of claim 1, wherein the shaft is made from a transparent material.

11. The device of claim 1, wherein the housing further comprises a slot for the insertion of a memory device.

12. The device of claim 11, wherein the memory device is selected from the group consisting of an SD card, a micro-SD card, a USB device and a flash drive.

13. A system for wireless observation of a target tissue, comprising:
the wireless endoscopic viewing device of claim 1; and
an external receiver for receiving images transmitted by the wireless endoscopic viewing device.

14. The system of claim 13, wherein the external wireless receiver is selected from the group consisting of monitors, computer terminals comprising a monitor, smart phones and tablet computers.

15. The system of claim 13, wherein a function of the wireless endoscopic viewing device is controlled with a dedicated application (app) installed or resident on the external wireless receiver.

16. The system of claim 13, wherein the external receiver comprises:
an antenna for receiving images transmitted by the wireless endoscopic viewing device; and
a video output to be connected to a video monitor.

17. A method for wireless observation of a target site in a subject in need thereof with the wireless endoscopic viewing device of claim 1 through, comprising:
establishing an entry portal having access to the target site,
inserting the distal end of the disposable cannula through the entry portal,
advancing the cannula toward the target site; and
imaging the target site with the camera.

18. A kit for wireless observation of a target site in a subject comprising the wireless endoscopic viewing device of claim 1.

19. The kit of claim 18, further comprising a receiver.

20. The kit of claim 18, further comprising an obturator.
